(19) United States
(12) Patent Application Publication
(10) Pub. No.: US 2022/0084649 A1
(43) Pub. Date: Mar. 17, 2022

(54) INFUSION SYSTEMS AND METHODS FOR PATIENT PREDICTIONS USING ASSOCIATION MINING

(71) Applicant: MEDTRONIC MINIMED, INC., Northridge, CA (US)

(72) Inventors: Daniel Anderson, Raanana (IL); Itay Ben Shushan, Rehovot (IL); Yaron Hadad, San Francisco, CA (US); Daniel Modlinger, Tel Aviv (IL); Noa Tsror, Tel Aviv (IL); Tal Elgali, Karmiel (IL)

(21) Appl. No.: 17/024,557

(22) Filed: Sep. 17, 2020

(51) Int. Cl. G16H 20/17; G16H 50/20; G16H 10/60

(52) U.S. Cl. CPC G16H 20/17; G16H 10/60; G16H 50/20

(57) ABSTRACT

Patient monitoring systems and related devices and operating methods are provided. One exemplary method involves obtaining a predictive association model determined based on historical data associated with the patient that includes a plurality of associations, obtaining real-time data associated with the patient from a plurality of different data sources, determining a current state of the patient based at least in part on the real-time data, predicting occurrence of an event when the current state of the patient corresponds to an association of the plurality of associations, and in response to predicting the occurrence of the event, initiating an action in response to the predicted event.

### TECHNICAL FIELD

Embodiments of the subject matter described herein relate generally to medical devices, and more particularly, embodiments of the subject matter relate to automatically adapting operations of a fluid infusion device in a personalized manner.

### BACKGROUND

Infusion pump devices and systems are relatively well known in the medical arts, for use in delivering or dispensing an agent, such as insulin or another prescribed medication, to a patient. A typical infusion pump includes a pump drive system which typically includes a small motor and drive train components that convert rotational motor motion to a translational displacement of a plunger (or stopper) in a reservoir that delivers medication from the reservoir to the body of a user via a fluid path created between the reservoir and the body of a user. Use of infusion pump therapy has been increasing, especially for delivering insulin for diabetics.

Continuous insulin infusion provides greater control of a diabetic's condition, and hence, control schemes are being developed that allow insulin infusion pumps to monitor and regulate a patient's blood glucose level in a substantially continuous and autonomous manner, for example, overnight while the user is sleeping. Regulating blood glucose level is complicated by variations in the response time for the type of insulin being used along with each patient's individual insulin response. Furthermore, a patient's daily activities and experiences may cause that patient's insulin response to vary throughout the course of a day or from one day to the next. Thus, it is desirable to account for the anticipated variations or fluctuations in the patient's insulin response caused by the patient's activities or other condition(s) experienced by the patient.

Managing a diabetic's blood glucose level is also complicated by the patient's consumption of meals or carbohydrates. Often, a user manually administers a bolus of insulin at or around mealtime to mitigate postprandial hyperglycemia. To effectively mitigate postprandial hyperglycemia while also avoiding postprandial hypoglycemia, the user is often required to estimate the amount of carbohydrates being consumed, with that amount of carbohydrates then being utilized to determine the appropriate bolus dosage. While undesirably increasing the burden on the patient for managing his or her therapy, manual errors such as miscounting carbohydrates or failing to initiate a bolus in a timely manner can also reduce the therapy effectiveness. Accordingly, there is a need facilitate improved glucose control that reduces the likelihood of manual errors while also reducing patient workload.

### BRIEF SUMMARY

An embodiment of a method of monitoring a physiological condition of a patient is provided. The method involves obtaining a predictive association model associated with the patient, wherein the predictive association model is determined based on historical data associated with the patient and comprises a plurality of associations, obtaining real-time data associated with the patient from a plurality of different data sources, determining a current state of the patient based at least in part on the real-time data, predicting occurrence of an event when the current state of the patient corresponds to an association of the plurality of associations, and in response to predicting the occurrence of the event, initiating an action in response to the predicted event.

In another embodiment, a method of monitoring a physiological condition of the patient in connection with operation of an infusion device capable of delivering fluid to the patient to influence the physiological condition of the patient involves predicting an occurrence of an event based at least in part on real-time data associated with the patient using a predictive association model associated with the patient when a current state of the patient indicated by the real-time data matches a patient state associated with a respective contextual state association of a plurality of contextual state associations associated with the predictive association model, and in response to the predicted occurrence of the event, automatically initiating one or more actions influenced by the event.

In another embodiment, a system is provided that includes a database, a server in communication with the database, and a device associated with a patient. The database maintains historical data associated with the patient. The server is configured to transform real values of the historical data to categorical values for a plurality of fields of patient data and determine a predictive association model for the patient based on relationships between respective categorical values for respective subsets of the plurality of fields of patient data and historical occurrences of an event. The predictive association model comprises a plurality of contextual state associations and each contextual state association comprises a respective subset of the categorical state values for a respective subset of the plurality of fields of patient data. The device obtains the predictive association model from the server via a network, predicts occurrence of the event when real-time data associated with the patient corresponds to a respective contextual state association of the plurality of contextual state associations associated with the predictive association model, and initiates an action in response to predicting the event.

In some embodiments, a non-transitory computer-readable medium is provided having instructions stored thereon that are executable by a control system associated with a medical device to obtain a predictive association model associated with a patient, wherein the predictive association model is determined based on historical data associated with the patient and comprises a plurality of associations, obtain real-time data associated with the patient from a plurality of different data sources, determine a current state of the patient based at least in part on the real-time data, predict occurrence of an event when the current state of the patient corresponds to an association of the plurality of associations, and in response to predicting the occurrence of the event, initiate an action by the medical device in response to the predicted event. In one or more embodiments, the medical device is an infusion device, wherein the instructions are configurable to cause the infusion device to automatically initiate fluid delivery in response to the predicted event.

This summary is provided to introduce a selection of concepts in a simplified form that are further described below in the detailed description. This summary is not intended to identify key features or essential features of the claimed subject matter, nor is it intended to be used as an aid in determining the scope of the claimed subject matter.

### BRIEF DESCRIPTION OF THE DRAWINGS

A more complete understanding of the subject matter may be derived by referring to the detailed description and claims when considered in conjunction with the following figures, wherein like reference numbers refer to similar elements throughout the figures, which may be illustrated for simplicity and clarity and are not necessarily drawn to scale.

FIG. 1 depicts an exemplary embodiment of an infusion system;

FIG. 2 is a block diagram of an exemplary infusion system suitable for use with a fluid infusion device in one or more embodiments;

FIG. 3 is a block diagram of an exemplary pump control system suitable for use in the infusion device in the infusion system of FIG. 2 in one or more embodiments;

FIG. 4 is a block diagram of a closed-loop control system that may be implemented or otherwise supported by the pump control system in the fluid infusion device of FIGS. 2-3 in one or more exemplary embodiments;

FIG. 5 is a block diagram of an exemplary patient monitoring system;

FIG. 6 is a flow diagram of an exemplary association modeling process in accordance with one or more exemplary embodiments;

FIG. 7 depicts an example merging of different data items into a common data entry in connection with an exemplary embodiment of the association modeling process of FIG. 6;

FIG. 8 depicts an example autopopulating of a portion of a patient data table in connection with an exemplary embodiment of the association modeling process of FIG. 6;

FIG. 9 depicts an example historical contextual table data entry that may be generated from a portion of a patient data table in connection with an exemplary embodiment of the association modeling process of FIG. 6;

FIG. 10 depicts an example hierarchical clustering and categorization of real data values in connection with an exemplary embodiment of the association modeling process of FIG. 6;

FIG. 11 depicts an example Boolean transformation of categorical data values in connection with an exemplary embodiment of the association modeling process of FIG. 6;

FIG. 12 depicts an example co-occurrence table suitable for use in connection with an exemplary embodiment of the association modeling process of FIG. 6;

FIG. 13 depicts an exemplary event prediction process suitable for implementation in connection with the association modeling process of FIG. 6 to predict occurrence of an event using a patient-specific predictive association model; and

FIG. 14 depicts an exemplary ontological prediction process for predicting occurrence of an event in one or more exemplary embodiments.

### DETAILED DESCRIPTION

The following detailed description is merely illustrative in nature and is not intended to limit the embodiments of the subject matter or the application and uses of such embodiments. As used herein, the word “exemplary” means “serving as an example, instance, or illustration.” Any implementation described herein as exemplary is not necessarily to be construed as preferred or advantageous over other implementations. Furthermore, there is no intention to be bound by any expressed or implied theory presented in the preceding technical field, background, brief summary or the following detailed description.

Exemplary embodiments of the subject matter described herein are implemented in conjunction with medical devices, such as portable electronic medical devices. Although many different applications are possible, the following description may focus on embodiments that incorporate a fluid infusion device (or infusion pump) as part of an infusion system deployment. That said, the subject matter described herein is not limited to infusion devices (or any particular configuration or realization thereof) and may be implemented in an equivalent manner in the context of multiple daily injection (MDI) therapy regimen or other medical devices, such as continuous glucose monitoring (CGM) devices, injection pens (e.g., smart injection pens), and the like. For the sake of brevity, conventional techniques related to infusion system operation, insulin pump and/or infusion set operation, and other functional aspects of the systems (and the individual operating components of the systems) may not be described in detail here. Examples of infusion pumps may be of the type described in, but not limited to, U.S. Pat. Nos. 4,562,751; 4,685,903; 5,080,653; 5,505,709; 5,097,122; 6,485,465; 6,554,798; 6,558,320; 6,558,351; 6,641,533; 6,659,980; 6,752,787; 6,817,990; 6,932,584; and 7,621,893; each of which are herein incorporated by reference.

Embodiments of the subject matter described herein generally relate to fluid infusion devices including a motor or other actuation arrangement that is operable to linearly displace a plunger (or stopper) of a reservoir provided within the fluid infusion device to deliver a dosage of fluid, such as insulin, to the body of a patient. In one or more exemplary embodiments, delivery commands (or dosage commands) that govern operation of the motor are determined based on a difference between a measured value for a physiological condition in the body of the patient and a target value using closed-loop control to regulate the measured value to the target value. As described in greater detail below, in one or more embodiments, a meal, exercise, or other activity or event that is likely to influence the patient's response (or sensitivity) to the fluid being administered is predicted, detected or otherwise identified, so that delivery commands for operating the infusion device may be adjusted to account for the anticipated change in the patient's glycemic condition.

For purposes of explanation, the subject matter may be described herein primarily in the context of identifying or detecting a meal for purposes of regulating a glucose level in the body of the patient by administering dosages of insulin that account for the meal in a personalized manner. That said, the subject matter described herein is not necessarily limited to glucose regulation, insulin infusion, or meals, and in practice, could be implemented in an equivalent manner with respect to other medications, physiological conditions, exercise or other activities, and/or the like.

As described in greater detail below, in exemplary embodiments, a patient's historical data is analyzed to identify associations of concurrent contextual variable states that are correlative to and predictive of the occurrence of an event of interest. For example, a patient's historical data may be analyzed to identify different sets of concurrent contextual variable states that are correlative to meals that occurred concurrently or contemporaneously. A predictive association model is derived that includes an optimal set of concurrent contextual variable state associations that are correlative to and predictive of the event of interest. In this regard, the patient's historical data is analyzed to harvest associations between taxonomies of various events for the patient, for example, by mapping each meal that was consumed by the patient to a particular ontology for which statistical correlations may be calculated between different levels in the ontology and other events that preceded it. Thereafter, real-time data associated with the patient may be analyzed to detect or otherwise identify substantially in real-time when an aspect of the current patient context matches or otherwise corresponds to a contextual state association assigned to the patient's predictive association model. In this regard, measurement data, location data, network connectivity data, activity data, and/or other contextual data may be passively collected from a patient's mobile device(s), medical device(s), and/or other wearable devices and analyzed in real-time to identify when the patient is engaged in behavioral pattern that has historically been correlative to and predictive of occurrence of the event.

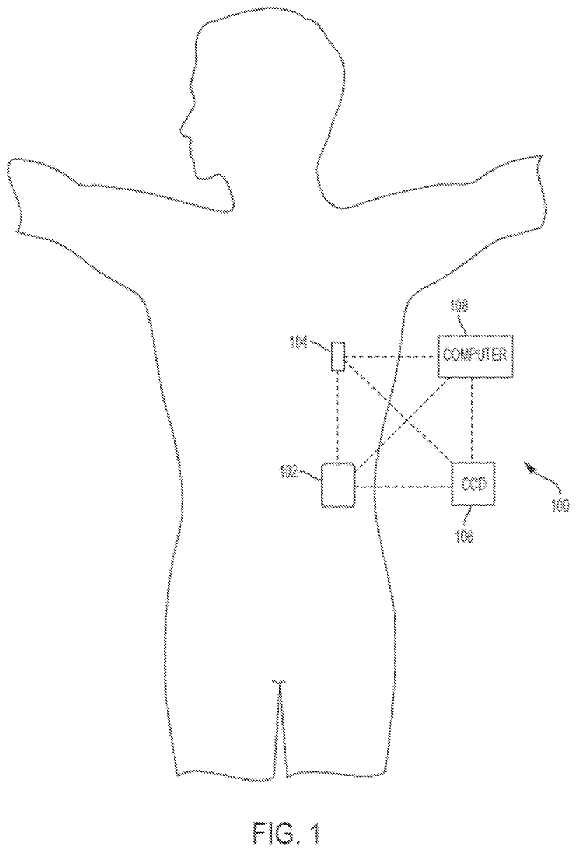

Turning now to FIG. 1, one exemplary embodiment of an infusion system 100 includes, without limitation, a fluid infusion device (or infusion pump) 102, a sensing arrangement 104, a command control device (CCD) 106, and a computer 108. The components of an infusion system 100 may be realized using different platforms, designs, and configurations, and the embodiment shown in FIG. 1 is not exhaustive or limiting. In practice, the infusion device 102 and the sensing arrangement 104 are secured at desired locations on the body of a user (or patient), as illustrated in FIG. 1. In this regard, the locations at which the infusion device 102 and the sensing arrangement 104 are secured to the body of the user in FIG. 1 are provided only as a representative, non-limiting, example. The elements of the infusion system 100 may be similar to those described in U.S. Pat. No. 8,674,288, the subject matter of which is hereby incorporated by reference in its entirety.

In the illustrated embodiment of FIG. 1, the infusion device 102 is designed as a portable medical device suitable for infusing a fluid, a liquid, a gel, or other medicament into the body of a user. In exemplary embodiments, the infused fluid is insulin, although many other fluids may be administered through infusion such as, but not limited to, HIV drugs, drugs to treat pulmonary hypertension, iron chelation drugs, pain medications, anti-cancer treatments, medications, vitamins, hormones, or the like. In some embodiments, the fluid may include a nutritional supplement, a dye, a tracing medium, a saline medium, a hydration medium, or the like.

The sensing arrangement 104 generally represents the components of the infusion system 100 configured to sense, detect, measure or otherwise quantify a condition of the user, and may include a sensor, a monitor, or the like, for providing data indicative of the condition that is sensed, detected, measured or otherwise monitored by the sensing arrangement. In this regard, the sensing arrangement 104 may include electronics and enzymes reactive to a biological condition, such as a blood glucose level, or the like, of the user, and provide data indicative of the blood glucose level to the infusion device 102, the CCD 106 and/or the computer 108. For example, the infusion device 102, the CCD 106 and/or the computer 108 may include a display for presenting information or data to the user based on the sensor data received from the sensing arrangement 104, such as, for example, a current glucose level of the user, a graph or chart of the patient's glucose level versus time, device status indicators, alert messages, or the like. In other embodiments, the infusion device 102, the CCD 106 and/or the computer 108 may include electronics and software that are configured to analyze sensor data and operate the infusion device 102 to deliver fluid to the body of the user based on the sensor data and/or preprogrammed delivery routines. Thus, in exemplary embodiments, one or more of the infusion device 102, the sensing arrangement 104, the CCD 106, and/or the computer 108 includes a transmitter, a receiver, and/or other transceiver electronics that allow for communication with other components of the infusion system 100, so that the sensing arrangement 104 may transmit sensor data or monitor data to one or more of the infusion device 102, the CCD 106 and/or the computer 108.

Still referring to FIG. 1, in various embodiments, the sensing arrangement 104 may be secured to the body of the user or embedded in the body of the user at a location that is remote from the location at which the infusion device 102 is secured to the body of the user. In various other embodiments, the sensing arrangement 104 may be incorporated within the infusion device 102. In other embodiments, the sensing arrangement 104 may be separate and apart from the infusion device 102, and may be, for example, part of the CCD 106. In such embodiments, the sensing arrangement 104 may be configured to receive a biological sample, analyte, or the like, to measure a condition of the user.

In some embodiments, the CCD 106 and/or the computer 108 may include electronics and other components configured to perform processing, delivery routine storage, and to control the infusion device 102 in a manner that is influenced by sensor data measured by and/or received from the sensing arrangement 104. By including control functions in the CCD 106 and/or the computer 108, the infusion device 102 may be made with more simplified electronics. However, in other embodiments, the infusion device 102 may include all control functions, and may operate without the CCD 106 and/or the computer 108. In various embodiments, the CCD 106 may be a portable electronic device. In addition, in various embodiments, the infusion device 102 and/or the sensing arrangement 104 may be configured to transmit data to the CCD 106 and/or the computer 108 for display or processing of the data by the CCD 106 and/or the computer 108.

In some embodiments, the CCD 106 and/or the computer 108 may provide information to the user that facilitates the patient's subsequent use of the infusion device 102. For example, the CCD 106 may provide information to the user to allow the user to determine the rate or dose of medication to be administered into the patient's body. In other embodiments, the CCD 106 may provide information to the infusion device 102 to autonomously control the rate or dose of medication administered into the body of the user. In some embodiments, the sensing arrangement 104 may be integrated into the CCD 106. Such embodiments may allow the user to monitor a condition by providing, for example, a sample of his or her blood to the sensing arrangement 104 to assess his or her condition. In some embodiments, the sensing arrangement 104 and the CCD 106 may be used for determining glucose levels in the blood and/or body fluids of the user without the use of, or necessity of, a wire or cable connection between the infusion device 102 and the sensing arrangement 104 and/or the CCD 106.

In some embodiments, the sensing arrangement 104 and/or the infusion device 102 are cooperatively configured to utilize a closed-loop system for delivering fluid to the user. Examples of sensing devices and/or infusion pumps utilizing closed-loop systems may be found at, but are not limited to, the following U.S. Pat. Nos. 6,088,608, 6,119,028, 6,589,229, 6,740,072, 6,827,702, 7,323,142, and 7,402,153 or United States Patent Application Publication No. 2014/0066889, all of which are incorporated herein by reference in their entirety. In such embodiments, the sensing arrangement 104 is configured to sense or measure a condition of the user, such as, blood glucose level or the like. The infusion device 102 is configured to deliver fluid in response to the condition sensed by the sensing arrangement 104. In turn, the sensing arrangement 104 continues to sense or otherwise quantify a current condition of the user, thereby allowing the infusion device 102 to deliver fluid continuously in response to the condition currently (or most recently) sensed by the sensing arrangement 104 indefinitely. In some embodiments, the sensing arrangement 104 and/or the infusion device 102 may be configured to utilize the closed-loop system only for a portion of the day, for example only when the user is asleep or awake.

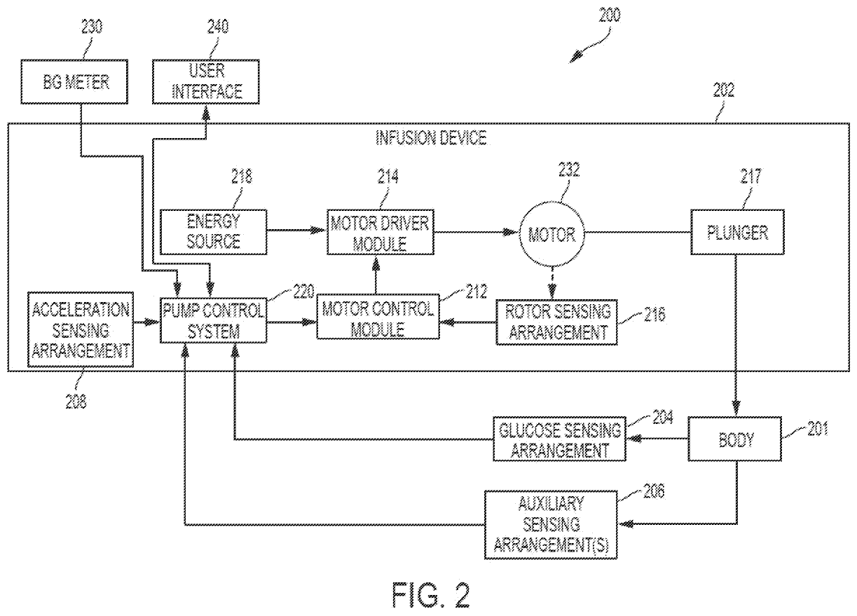

FIG. 2 depicts an exemplary embodiment of an infusion system 200 suitable for use with an infusion device 202, such as the infusion device 102 described above. The infusion system 200 is capable of controlling or otherwise regulating a physiological condition in the body 201 of a user to a desired (or target) value or otherwise maintain the condition within a range of acceptable values in an automated or autonomous manner. In one or more exemplary embodiments, the condition being regulated is sensed, detected, measured or otherwise quantified by a sensing arrangement 204 (e.g., sensing arrangement 104) communicatively coupled to the infusion device 202. However, it should be noted that in alternative embodiments, the condition being regulated by the infusion system 200 may be correlative to the measured values obtained by the sensing arrangement 204. That said, for clarity and purposes of explanation, the subject matter may be described herein in the context of the sensing arrangement 204 being realized as a glucose sensing arrangement that senses, detects, measures or otherwise quantifies the patient's glucose level, which is being regulated in the body 201 of the user by the infusion system 200.

In exemplary embodiments, the sensing arrangement 204 includes one or more interstitial glucose sensing elements that generate or otherwise output electrical signals (alternatively referred to herein as measurement signals) having a signal characteristic that is correlative to, influenced by, or otherwise indicative of the relative interstitial fluid glucose level in the body 201 of the user. The output electrical signals are filtered or otherwise processed to obtain a measurement value indicative of the patient's interstitial fluid glucose level. In exemplary embodiments, a blood glucose meter 230, such as a finger stick device, is utilized to directly sense, detect, measure or otherwise quantify the blood glucose in the body 201 of the user. In this regard, the blood glucose meter 230 outputs or otherwise provides a measured blood glucose value that may be utilized as a reference measurement for calibrating the sensing arrangement 204 and converting a measurement value indicative of the patient's interstitial fluid glucose level into a corresponding calibrated blood glucose value. For purposes of explanation, the calibrated blood glucose value calculated based on the electrical signals output by the sensing element(s) of the sensing arrangement 204 may alternatively be referred to herein as the sensor glucose value, the sensed glucose value, or variants thereof.

In exemplary embodiments, the infusion system 200 also includes one or more additional sensing arrangements 206, 208 configured to sense, detect, measure or otherwise quantify a characteristic of the body 201 of the user that is indicative of a condition in the body 201 of the user. In this regard, in addition to the glucose sensing arrangement 204, one or more auxiliary sensing arrangements 206 may be worn, carried, or otherwise associated with the body 201 of the user to measure characteristics or conditions of the patient (or the patient's activity) that may influence the patient's glucose levels or insulin sensitivity. For example, a heart rate sensing arrangement 206 could be worn on or otherwise associated with the patient's body 201 to sense, detect, measure or otherwise quantify the patient's heart rate, which, in turn, may be indicative of exercise (and the intensity thereof) that is likely to influence the patient's glucose levels or insulin response in the body 201. In yet another embodiment, another invasive, interstitial, or subcutaneous sensing arrangement 206 may be inserted into the body 201 of the user to obtain measurements of another physiological condition that may be indicative of exercise (and the intensity thereof), such as, for example, a lactate sensor, a ketone sensor, or the like. Depending on the embodiment, the auxiliary sensing arrangement(s) 206 could be realized as a standalone component worn by the user, or alternatively, the auxiliary sensing arrangement(s) 206 may be integrated with the infusion device 202 or the glucose sensing arrangement 204.

The illustrated infusion system 200 also includes an acceleration sensing arrangement 208 (or accelerometer) that may be worn on or otherwise associated with the patient's body 201 to sense, detect, measure or otherwise quantify an acceleration of the patient's body 201, which, in turn, may be indicative of exercise or some other condition in the body 201 that is likely to influence the patient's insulin response. While the acceleration sensing arrangement 208 is depicted as being integrated into the infusion device 202 in FIG. 2, in alternative embodiments, the acceleration sensing arrangement 208 may be integrated with another sensing arrangement 204, 206 on the body 201 of the user, or the acceleration sensing arrangement 208 may be realized as a separate standalone component that is worn by the user.

In some embodiments, the infusion device 202 (or the infusion system 200) may also include one or more environmental sensing arrangements to sense, detect, measure or otherwise quantify the current operating environment around the infusion device 202. In this regard, the environmental sensing arrangements may include one or more of a temperature sensing arrangement (or thermometer), a humidity sensing arrangement, a pressure sensing arrangement (or barometer), and/or the like. Additionally, the infusion device 202 (or the infusion system 200) may also include a position sensing arrangement to sense, detect, measure or otherwise quantify the current geographic location of the infusion device 202, such as, for example, a global positioning system (GPS) receiver. Again, it should be noted that such sensing arrangements could be integrated into the infusion device 202, integrated with other components, or realized as separate standalone components that are worn or carried by the patient.

In the illustrated embodiment, the pump control system 220 generally represents the electronics and other components of the infusion device 202 that control operation of the fluid infusion device 202 according to a desired infusion delivery program in a manner that is influenced by the sensed glucose value indicating the current glucose level in the body 201 of the user. For example, to support a closed-loop operating mode, the pump control system 220 maintains, receives, or otherwise obtains a target or commanded glucose value, and automatically generates or otherwise determines dosage commands for operating an actuation arrangement, such as a motor 232, to displace the plunger 217 and deliver insulin to the body 201 of the user based on the difference between the sensed glucose value and the target glucose value. In other operating modes, the pump control system 220 may generate or otherwise determine dosage commands configured to maintain the sensed glucose value below an upper glucose limit, above a lower glucose limit, or otherwise within a desired range of glucose values. In practice, the infusion device 202 may store or otherwise maintain the target value, upper and/or lower glucose limit(s), insulin delivery limit(s), and/or other glucose threshold value(s) in a data storage element accessible to the pump control system 220. As described in greater detail, in one or more exemplary embodiments, the pump control system 220 automatically adjusts or adapts one or more parameters or other control information used to generate commands for operating the motor 232 in a manner that accounts for a likely change in the patient's glucose level or insulin response resulting from a meal, exercise, or other activity.

Still referring to FIG. 2, the target glucose value and other threshold glucose values utilized by the pump control system 220 may be received from an external component (e.g., CCD 106 and/or computing device 108) or be input by a user via a user interface element 240 associated with the infusion device 202. In practice, the one or more user interface element(s) 240 associated with the infusion device 202 typically include at least one input user interface element, such as, for example, a button, a keypad, a keyboard, a knob, a joystick, a mouse, a touch panel, a touchscreen, a microphone or another audio input device, and/or the like. Additionally, the one or more user interface element(s) 240 include at least one output user interface element, such as, for example, a display element (e.g., a light-emitting diode or the like), a display device (e.g., a liquid crystal display or the like), a speaker or another audio output device, a haptic feedback device, or the like, for providing notifications or other information to the user. It should be noted that although FIG. 2 depicts the user interface element(s) 240 as being separate from the infusion device 202, in practice, one or more of the user interface element(s) 240 may be integrated with the infusion device 202. Furthermore, in some embodiments, one or more user interface element(s) 240 are integrated with the sensing arrangement 204 in addition to and/or in alternative to the user interface element(s) 240 integrated with the infusion device 202. The user interface element(s) 240 may be manipulated by the user to operate the infusion device 202 to deliver correction boluses, adjust target and/or threshold values, modify the delivery control scheme or operating mode, and the like, as desired.

Still referring to FIG. 2, in the illustrated embodiment, the infusion device 202 includes a motor controller 212 coupled to a motor 232 (e.g., motor assembly 207) that is operable to displace a plunger 217 (e.g., plunger 217) in a reservoir (e.g., reservoir 205) and provide a desired amount of fluid to the body 201 of a user. In this regard, displacement of the plunger 217 results in the delivery of a fluid, such as insulin, that is capable of influencing the patient's physiological condition to the body 201 of the user via a fluid delivery path (e.g., via tubing of an infusion set). A motor driver 214 is coupled between an energy source 218 and the motor 232. The motor controller 212 is coupled to the motor driver 214, and the motor controller 212 generates or otherwise provides command signals that operate the motor driver 214 to provide current (or power) from the energy source 218 to the motor 232 to displace the plunger 217 in response to receiving, from a pump control system 220, a dosage command indicative of the desired amount of fluid to be delivered.

In exemplary embodiments, the energy source 218 is realized as a battery housed within the infusion device 202 (e.g., within the infusion device housing) that provides direct current (DC) power. In this regard, the motor driver 214 generally represents the combination of circuitry, hardware and/or other electrical components configured to convert or otherwise transfer DC power provided by the energy source 218 into alternating electrical signals applied to respective phases of the stator windings of the motor 232 that result in current flowing through the stator windings that generates a stator magnetic field and causes the rotor of the motor 232 to rotate. The motor controller 212 is configured to receive or otherwise obtain a commanded dosage from the pump control system 220, convert the commanded dosage to a commanded translational displacement of the plunger 217, and command, signal, or otherwise operate the motor driver 214 to cause the rotor of the motor 232 to rotate by an amount that produces the commanded translational displacement of the plunger 217. For example, the motor controller 212 may determine an amount of rotation of the rotor required to produce translational displacement of the plunger 217 that achieves the commanded dosage received from the pump control system 220. Based on the current rotational position (or orientation) of the rotor with respect to the stator that is indicated by the output of the rotor sensing arrangement 216, the motor controller 212 determines the appropriate sequence of alternating electrical signals to be applied to the respective phases of the stator windings that should rotate the rotor by the determined amount of rotation from its current position (or orientation). In embodiments where the motor 232 is realized as a BLDC motor, the alternating electrical signals commutate the respective phases of the stator windings at the appropriate orientation of the rotor magnetic poles with respect to the stator and in the appropriate order to provide a rotating stator magnetic field that rotates the rotor in the desired direction. Thereafter, the motor controller 212 operates the motor driver 214 to apply the determined alternating electrical signals (e.g., the command signals) to the stator windings of the motor 232 to achieve the desired delivery of fluid to the user.

When the motor controller 212 is operating the motor driver 214, current flows from the energy source 218 through the stator windings of the motor 232 to produce a stator magnetic field that interacts with the rotor magnetic field. In some embodiments, after the motor controller 212 operates the motor driver 214 and/or motor 232 to achieve the commanded dosage, the motor controller 212 ceases operating the motor driver 214 and/or motor 232 until a subsequent dosage command is received. In this regard, the motor driver 214 and the motor 232 enter an idle state during which the motor driver 214 effectively disconnects or isolates the stator windings of the motor 232 from the energy source 218. In other words, current does not flow from the energy source 218 through the stator windings of the motor 232 when the motor 232 is idle, and thus, the motor 232 does not consume power from the energy source 218 in the idle state, thereby improving efficiency.

Depending on the embodiment, the motor controller 212 may be implemented or realized with a general purpose processor, a microprocessor, a controller, a microcontroller, a state machine, a content addressable memory, an application specific integrated circuit, a field programmable gate array, any suitable programmable logic device, discrete gate or transistor logic, discrete hardware components, or any combination thereof, designed to perform the functions described herein. In exemplary embodiments, the motor controller 212 includes or otherwise accesses a data storage element or memory, including any sort of random access memory (RAM), read only memory (ROM), flash memory, registers, hard disks, removable disks, magnetic or optical mass storage, or any other short or long term storage media or other non-transitory computer-readable medium, which is capable of storing programming instructions for execution by the motor controller 212. The computer-executable programming instructions, when read and executed by the motor controller 212, cause the motor controller 212 to perform or otherwise support the tasks, operations, functions, and processes described herein.

It should be appreciated that FIG. 2 is a simplified representation of the infusion device 202 for purposes of explanation and is not intended to limit the subject matter described herein in any way. In this regard, depending on the embodiment, some features and/or functionality of the sensing arrangement 204 may implemented by or otherwise integrated into the pump control system 220, or vice versa. Similarly, in practice, the features and/or functionality of the motor controller 212 may implemented by or otherwise integrated into the pump control system 220, or vice versa. Furthermore, the features and/or functionality of the pump control system 220 may be implemented by control electronics located in the housing of the fluid infusion device 202, while in alternative embodiments, the pump control system 220 may be implemented by a remote computing device that is physically distinct and/or separate from the infusion device 202, such as, for example, the CCD 106 or the computing device 108.

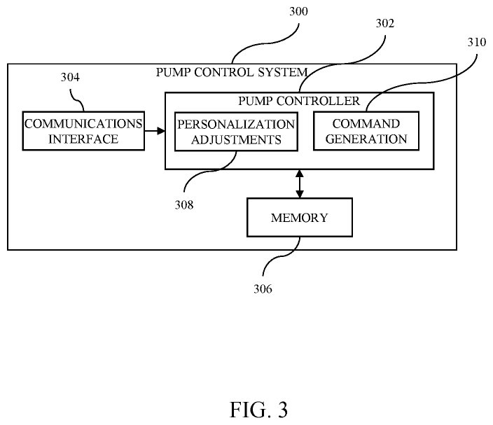

FIG. 3 depicts an exemplary embodiment of a pump control system 300 suitable for use as the pump control system 220 in FIG. 2 in accordance with one or more embodiments. The illustrated pump control system 300 includes, without limitation, a pump controller 302, a communications interface 304, and a data storage element (or memory) 306. The pump controller 302 is coupled to the communications interface 304 and the memory 306, and the pump controller 302 is suitably configured to support the operations, tasks, and/or processes described herein. In various embodiments, the pump controller 302 is also coupled to one or more user interface elements (e.g., user interface 240) for receiving user inputs (e.g., target glucose values or other glucose thresholds) and providing notifications, alerts, or other therapy information to the user.

The communications interface 304 generally represents the hardware, circuitry, logic, firmware and/or other components of the pump control system 300 that are coupled to the pump controller 302 and configured to support communications between the pump control system 300 and the various sensing arrangements 204, 206, 208. In this regard, the communications interface 304 may include or otherwise be coupled to one or more transceivers capable of supporting wireless communications between the pump control system 220, 300 and the sensing arrangement 204, 206, 208. For example, the communications interface 304 may be utilized to receive sensor measurement values or other measurement data from each sensing arrangement 204, 206, 208 in an infusion system 200. In other embodiments, the communications interface 304 may be configured to support wired communications to/from the sensing arrangement(s) 204, 206, 208. In various embodiments, the communications interface 304 may also support communications with another electronic device (e.g., CCD 106 and/or computer 108) in an infusion system (e.g., to upload sensor measurement values to a server or other computing device, receive control information from a server or other computing device, and the like).

The pump controller 302 generally represents the hardware, circuitry, logic, firmware and/or other component of the pump control system 300 that is coupled to the communications interface 304 and configured to determine dosage commands for operating the motor 232 to deliver fluid to the body 201 based on measurement data received from the sensing arrangements 204, 206, 208 and perform various additional tasks, operations, functions and/or operations described herein. For example, in exemplary embodiments, pump controller 302 implements or otherwise executes a command generation application 310 that supports one or more autonomous operating modes and calculates or otherwise determines dosage commands for operating the motor 232 of the infusion device 202 in an autonomous operating mode based at least in part on a current measurement value for a condition in the body 201 of the user. For example, in a closed-loop operating mode, the command generation application 310 may determine a dosage command for operating the motor 232 to deliver insulin to the body 201 of the user based at least in part on the current glucose measurement value most recently received from the sensing arrangement 204 to regulate the patient's blood glucose level to a target reference glucose value. Additionally, the command generation application 310 may generate dosage commands for boluses that are manually initiated or otherwise instructed by a user via a user interface element.

In exemplary embodiments, the pump controller 302 also implements or otherwise executes a personalization application 308 that is cooperatively configured to interact with the command generation application 310 to support adjusting dosage commands or control information dictating the manner in which dosage commands are generated in a personalized, user-specific (or patient-specific) manner, as described in greater detail below. In this regard, in some embodiments, based on correlations between current or recent measurement data and the current operational context relative to historical data associated with the patient, the personalization application 308 may adjust or otherwise modify values for one or more parameters utilized by the command generation application 310 when determining dosage commands, for example, by modifying a parameter value at a register or location in memory 306 referenced by the command generation application 310. In yet other embodiments, the personalization application 308 may predict meals or other events or activities that are likely to be engaged in by the user and output or otherwise provide an indication of the predicted user behavior for confirmation or modification by the user, which, in turn, may then be utilized to adjust the manner in which dosage commands are generated to regulate glucose in a manner that accounts for the patient's behavior in a personalized manner.

Still referring to FIG. 3, depending on the embodiment, the pump controller 302 may be implemented or realized with a general purpose processor, a microprocessor, a controller, a microcontroller, a state machine, a content addressable memory, an application specific integrated circuit, a field programmable gate array, any suitable programmable logic device, discrete gate or transistor logic, discrete hardware components, or any combination thereof, designed to perform the functions described herein. In this regard, the steps of a method or algorithm described in connection with the embodiments disclosed herein may be embodied directly in hardware, in firmware, in a software module executed by the pump controller 302, or in any practical combination thereof. In exemplary embodiments, the pump controller 302 includes or otherwise accesses the data storage element or memory 306, which may be realized using any sort of non-transitory computer-readable medium capable of storing programming instructions for execution by the pump controller 302. The computer-executable programming instructions, when read and executed by the pump controller 302, cause the pump controller 302 to implement or otherwise generate the applications 308, 310 and perform tasks, operations, functions, and processes described herein.

It should be understood that FIG. 3 is a simplified representation of a pump control system 300 for purposes of explanation and is not intended to limit the subject matter described herein in any way. For example, in some embodiments, the features and/or functionality of the motor controller 212 may be implemented by or otherwise integrated into the pump control system 300 and/or the pump controller 302, for example, by the command generation application 310 converting the dosage command into a corresponding motor command, in which case, the separate motor controller 212 may be absent from an embodiment of the infusion device 202.

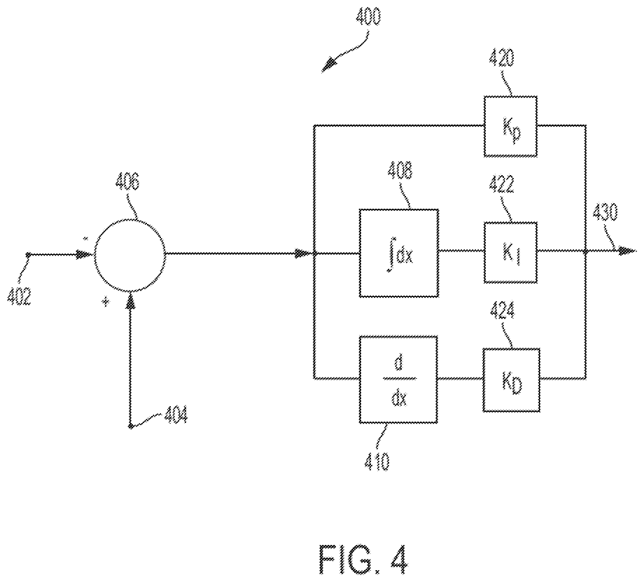

FIG. 4 depicts an exemplary closed-loop control system 400 that may be implemented by a pump control system 220, 300 to provide a closed-loop operating mode that autonomously regulates a condition in the body of a user to a reference (or target) value. It should be appreciated that FIG. 4 is a simplified representation of the control system 400 for purposes of explanation and is not intended to limit the subject matter described herein in any way.

In exemplary embodiments, the control system 400 receives or otherwise obtains a target glucose value at input 402. In some embodiments, the target glucose value may be stored or otherwise maintained by the infusion device 202 (e.g., in memory 306), however, in some alternative embodiments, the target value may be received from an external component (e.g., CCD 106 and/or computer 108). In one or more embodiments, the target glucose value may be calculated or otherwise determined prior to entering the closed-loop operating mode based on one or more patient-specific control parameters. For example, the target blood glucose value may be calculated based at least in part on a patient-specific reference basal rate and a patient-specific daily insulin requirement, which are determined based on historical delivery information over a preceding interval of time (e.g., the amount of insulin delivered over the preceding 24 hours). The control system 400 also receives or otherwise obtains a current glucose measurement value (e.g., the most recently obtained sensor glucose value) from the sensing arrangement 204 at input 404. The illustrated control system 400 implements or otherwise provides proportional-integral-derivative (PID) control to determine or otherwise generate delivery commands for operating the motor 232 based at least in part on the difference between the target glucose value and the current glucose measurement value. In this regard, the PID control attempts to minimize the difference between the measured value and the target value, and thereby regulates the measured value to the desired value. PID control parameters are applied to the difference between the target glucose level at input 402 and the measured glucose level at input 404 to generate or otherwise determine a dosage (or delivery) command provided at output 430. Based on that delivery command, the motor controller 212 operates the motor 232 to deliver insulin to the body of the user to influence the patient's glucose level, and thereby reduce the difference between a subsequently measured glucose level and the target glucose level.

The illustrated control system 400 includes or otherwise implements a summation block 406 configured to determine a difference between the target value obtained at input 402 and the measured value obtained from the sensing arrangement 204 at input 404, for example, by subtracting the target value from the measured value. The output of the summation block 406 represents the difference between the measured and target values, which is then provided to each of a proportional term path, an integral term path, and a derivative term path. The proportional term path includes a gain block 420 that multiplies the difference by a proportional gain coefficient, K_{P}, to obtain the proportional term. The integral term path includes an integration block 408 that integrates the difference and a gain block 422 that multiplies the integrated difference by an integral gain coefficient, KI, to obtain the integral term. The derivative term path includes a derivative block 410 that determines the derivative of the difference and a gain block 424 that multiplies the derivative of the difference by a derivative gain coefficient, K_{D}, to obtain the derivative term. The proportional term, the integral term, and the derivative term are then added or otherwise combined to obtain a delivery command that is utilized to operate the motor at output 430. Various implementation details pertaining to closed-loop PID control and determining gain coefficients are described in greater detail in U.S. Pat. No. 7,402,153, which is incorporated by reference.

In one or more exemplary embodiments, the PID gain coefficients are user-specific (or patient-specific) and dynamically calculated or otherwise determined prior to entering the closed-loop operating mode based on historical insulin delivery information (e.g., amounts and/or timings of previous dosages, historical correction bolus information, or the like), historical sensor measurement values, historical reference blood glucose measurement values, user-reported or user-input events (e.g., meals, exercise, and the like), and the like. In this regard, one or more patient-specific control parameters (e.g., an insulin sensitivity factor, a daily insulin requirement, an insulin limit, a reference basal rate, a reference fasting glucose, an active insulin action duration, pharmodynamical time constants, or the like) may be utilized to compensate, correct, or otherwise adjust the PID gain coefficients to account for various operating conditions experienced and/or exhibited by the infusion device 202. The PID gain coefficients may be maintained by the memory 306 accessible to the pump controller 302. In this regard, the memory 306 may include a plurality of registers associated with the control parameters for the PID control. For example, a first parameter register may store the target glucose value and be accessed by or otherwise coupled to the summation block 406 at input 402, and similarly, a second parameter register accessed by the proportional gain block 420 may store the proportional gain coefficient, a third parameter register accessed by the integration gain block 422 may store the integration gain coefficient, and a fourth parameter register accessed by the derivative gain block 424 may store the derivative gain coefficient.

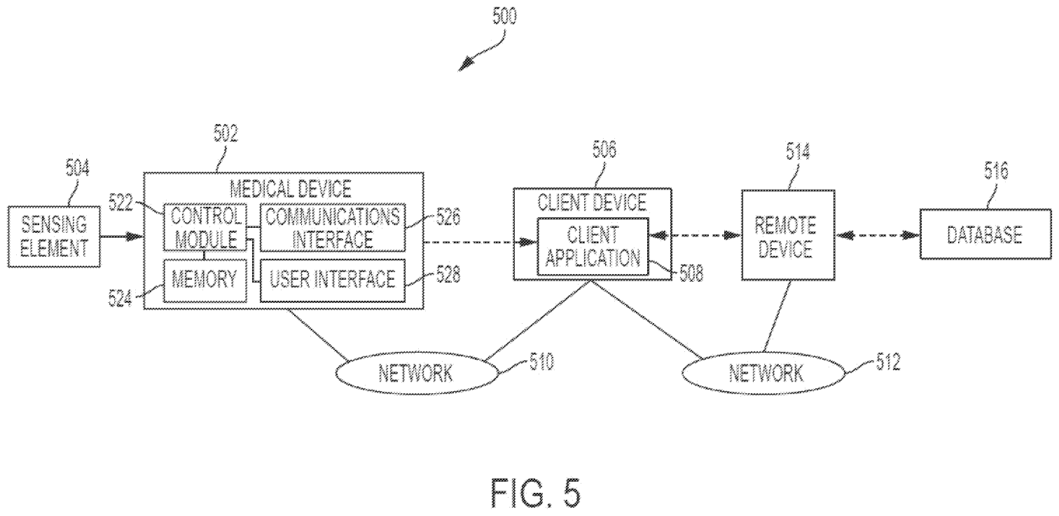

FIG. 5 depicts an exemplary embodiment of a patient monitoring system 500. The patient monitoring system 500 includes a medical device 502 that is communicatively coupled to a sensing element 504 that is inserted into the body of a patient or otherwise worn by the patient to obtain measurement data indicative of a physiological condition in the body of the patient, such as a sensed glucose level. The medical device 502 is communicatively coupled to a client device 506 via a communications network 510, with the client device 506 being communicatively coupled to a remote device 514 via another communications network 512. It should be noted that although FIG. 5 depicts a single remote device 514 for clarity and ease of illustration, in practice, the remote device 514 may be realized as a server farm or other combination of networked computing devices configured to provide or otherwise support a cloud computing environment or cloud-based applications. In this regard, the client device 506 may function as an intermediary for uploading or otherwise providing measurement data from the medical device 502 to the remote device 514. It should be appreciated that FIG. 5 depicts a simplified representation of a patient monitoring system 500 for purposes of explanation and is not intended to limit the subject matter described herein in any way.

In exemplary embodiments, the client device 506 is realized as a mobile phone, a smartphone, a tablet computer, or other similar mobile electronic device; however, in other embodiments, the client device 506 may be realized as any sort of electronic device capable of communicating with the medical device 502 via network 510, such as a laptop or notebook computer, a desktop computer, or the like. In exemplary embodiments, the network 510 is realized as a Bluetooth network, a ZigBee network, or another suitable personal area network. That said, in other embodiments, the network 510 could be realized as a wireless ad hoc network, a wireless local area network (WLAN), or local area network (LAN). The client device 506 includes or is coupled to a display device, such as a monitor, screen, or another conventional electronic display, capable of graphically presenting data and/or information pertaining to the physiological condition of the patient. The client device 506 also includes or is otherwise associated with a user input device, such as a keyboard, a mouse, a touchscreen, or the like, capable of receiving input data and/or other information from the user of the client device 506.

In exemplary embodiments, a user, such as the patient, the patient's doctor or another healthcare provider, or the like, manipulates the client device 506 to execute a client application 508 that supports communicating with the medical device 502 via the network 510. In this regard, the client application 508 supports establishing a communications session with the medical device 502 on the network 510 and receiving data and/or information from the medical device 502 via the communications session. The medical device 502 may similarly execute or otherwise implement a corresponding application or process that supports establishing the communications session with the client application 508. The client application 508 generally represents a process, service, or other software component that is executed, generated or otherwise implemented by the client device 506 to support the processes described herein. Accordingly, the client device 506 generally includes a processing system and a data storage element (or memory) capable of storing programming instructions for execution by the processing system, that, when read and executed, cause processing system to create, generate, or otherwise facilitate the client application 508 and perform or otherwise support the processes, tasks, operations, and/or functions described herein. Depending on the embodiment, the processing system may be implemented using any suitable processing system and/or device, such as, for example, one or more processors, central processing units (CPUs), controllers, microprocessors, microcontrollers, processing cores and/or other hardware computing resources configured to support the operation of the processing system described herein. Similarly, the data storage element or memory may be realized as a random access memory (RAM), read only memory (ROM), flash memory, magnetic or optical mass storage, or any other suitable non-transitory short or long term data storage or other computer-readable media, and/or any suitable combination thereof.

In one or more embodiments, the client device 506 and the medical device 502 establish an association (or pairing) with one another over the network 510 to support subsequently establishing a point-to-point communications session between the medical device 502 and the client device 506 via the network 510. For example, in accordance with one embodiment, the network 510 is realized as a Bluetooth network, wherein the medical device 502 and the client device 506 are paired with one another (e.g., by obtaining and storing network identification information for one another) by performing a discovery procedure or another suitable pairing procedure. The pairing information obtained during the discovery procedure allows either of the medical device 502 or the client device 506 to initiate the establishment of a secure communications session via the network 510.

In one or more exemplary embodiments, the client application 508 is also configured to store or otherwise maintain an address and/or other identification information for the remote device 514 on the second network 512. In this regard, the second network 512 may be physically and/or logically distinct from the network 510, such as, for example, the Internet, a cellular network, a wide area network (WAN), or the like. The remote device 514 generally represents a server or other computing device configured to receive and analyze or otherwise monitor measurement data, event log data, and potentially other information obtained for the patient associated with the medical device 502. In exemplary embodiments, the remote device 514 is coupled to a database 516 configured to store or otherwise maintain data associated with individual patients. In practice, the remote device 514 may reside at a location that is physically distinct and/or separate from the medical device 502 and the client device 506, such as, for example, at a facility that is owned and/or operated by or otherwise affiliated with a manufacturer of the medical device 502. For purposes of explanation, but without limitation, the remote device 514 may alternatively be referred to herein as a server.

It should be noted that in some embodiments, some or all of the functionality and processing intelligence of the remote computing device 514 can reside at the medical device 502 and/or at other components or computing devices that are compatible with the patient monitoring system 500. In other words, the patient monitoring system 500 need not rely on a network-based or a cloud-based server arrangement as depicted in FIG. 5, although such a deployment might be the most efficient and economical implementation. These and other alternative arrangements are contemplated by this disclosure. To this end, some embodiments of the system 500 may include additional devices and components that serve as data sources, data processing units, and/or recommendation delivery mechanisms. For example, the system 500 may include any or all of the following elements, without limitation: computer devices or systems; patient monitors; healthcare provider systems; data communication devices; and the like.

Still referring to FIG. 5, the sensing element 504 generally represents the component of the patient monitoring system 500 that is configured to generate, produce, or otherwise output one or more electrical signals indicative of a physiological condition that is sensed, measured, or otherwise quantified by the sensing element 504. In this regard, the physiological condition of a user influences a characteristic of the electrical signal output by the sensing element 504, such that the characteristic of the output signal corresponds to or is otherwise correlative to the physiological condition that the sensing element 504 is sensitive to. In exemplary embodiments, the sensing element 504 is realized as an interstitial glucose sensing element inserted at a location on the body of the patient that generates an output electrical signal having a current (or voltage) associated therewith that is correlative to the interstitial fluid glucose level that is sensed or otherwise measured in the body of the patient by the sensing element 504.

The medical device 502 generally represents the component of the patient monitoring system 500 that is communicatively coupled to the output of the sensing element 504 to receive or otherwise obtain the measurement data samples from the sensing element 504 (e.g., the measured glucose and characteristic impedance values), store or otherwise maintain the measurement data samples, and upload or otherwise transmit the measurement data to the server 514 via the client device 506. In one or more embodiments, the medical device 502 is realized as an infusion device 102, 202 configured to deliver a fluid, such as insulin, to the body of the patient. That said, in other embodiments, the medical device 502 could be a standalone sensing or monitoring device separate and independent from an infusion device (e.g., sensing arrangement 104, 204), such as, for example, a continuous glucose monitor (CGM), an interstitial glucose sensing arrangement, or similar device. It should be noted that although FIG. 5 depicts the medical device 502 and the sensing element 504 as separate components, in practice, the medical device 502 and the sensing element 504 may be integrated or otherwise combined to provide a unitary device that can be worn by the patient.

In exemplary embodiments, the medical device 502 includes a controller 522, a data storage element 524 (or memory), a communications interface 526, and a user interface 528. The user interface 528 generally represents the input user interface element(s) and/or output user interface element(s) associated with the medical device 502 (e.g., one or more user interface elements 240). The controller 522 generally represents the hardware, circuitry, logic, firmware and/or other component(s) of the medical device 502 that is coupled to the sensing element 504 to receive the electrical signals output by the sensing element 504 and perform or otherwise support various additional tasks, operations, functions and/or processes described herein. Depending on the embodiment, the controller 522 may be implemented or realized with a general purpose processor, a microprocessor, a controller, a microcontroller, a state machine, a content addressable memory, an application specific integrated circuit, a field programmable gate array, any suitable programmable logic device, discrete gate or transistor logic, discrete hardware components, or any combination thereof, designed to perform the functions described herein. In some embodiments, the controller 522 includes an analog-to-digital converter (ADC) or another similar sampling arrangement that samples or otherwise converts an output electrical signal received from the sensing element 504 into corresponding digital measurement data value. In other embodiments, the sensing element 504 may incorporate an ADC and output a digital measurement value.

The communications interface 526 generally represents the hardware, circuitry, logic, firmware and/or other components of the medical device 502 that are coupled to the controller 522 for outputting data and/or information from/to the medical device 502 to/from the client device 506. For example, the communications interface 526 may include or otherwise be coupled to one or more transceivers capable of supporting wireless communications between the medical device 502 and the client device 506. In exemplary embodiments, the communications interface 526 is realized as a Bluetooth transceiver or adapter configured to support Bluetooth Low Energy (BLE) communications.

In exemplary embodiments, the remote device 514 receives, from the client device 506, measurement data values associated with a particular patient (e.g., sensor glucose measurements, acceleration measurements, and the like) that were obtained using the sensing element 504, and the remote device 514 stores or otherwise maintains the historical measurement data in the database 516 in association with the patient (e.g., using one or more unique patient identifiers). Additionally, the remote device 514 may also receive, from or via the client device 506, meal data or other event log data that may be input or otherwise provided by the patient (e.g., via client application 508) and store or otherwise maintain historical meal data and other historical event or activity data associated with the patient in the database 516. In this regard, the meal data include, for example, a time or timestamp associated with a particular meal event, a meal type or other information indicative of the content or nutritional characteristics of the meal, and an indication of the size associated with the meal. In exemplary embodiments, the remote device 514 also receives historical fluid delivery data corresponding to basal or bolus dosages of fluid delivered to the patient by an infusion device 102, 202. For example, the client application 508 may communicate with an infusion device 102, 202 to obtain insulin delivery dosage amounts and corresponding timestamps from the infusion device 102, 202, and then upload the insulin delivery data to the remote device 514 for storage in association with the particular patient. The remote device 514 may also receive geolocation data and potentially other contextual data associated with a device 502, 506 from the client device 506 and/or client application 508, and store or otherwise maintain the historical operational context data in association with the particular patient. In this regard, one or more of the devices 502, 506 may include a global positioning system (GPS) receiver or similar hardware modules, components or circuitry capable of outputting or otherwise providing data characterizing the geographic location of the respective device 502, 506 in real-time.

The historical patient data may be analyzed by one or more of the remote device 514, the client device 506, and/or the medical device 502 to alter or adjust operation of an infusion device 102, 202 to influence fluid delivery in a personalized manner. For example, the patient's historical meal data and corresponding measurement data or other contextual data may be analyzed to predict a future time when the next meal is likely to be consumed by the patient, the likelihood of a future meal event within a specific time period, the likely size or amount of carbohydrates associated with a future meal, the likely type or nutritional content of the future meal, and/or the like. Moreover, the patient's historical measurement data for postprandial periods following historical meal events may be analyzed to model or otherwise characterize the patient's glycemic response to the predicted size and type of meal for the current context (e.g., time of day, day of week, geolocation, etc.). One or more aspects of the infusion device 102, 202 that control or regulate insulin delivery may then be modified or adjusted to proactively account for the patient's likely meal activity and glycemic response.

In one or more exemplary embodiments, the remote device 514 utilizes machine learning to determine which combination of historical sensor glucose measurement data, historical delivery data, historical auxiliary measurement data (e.g., historical acceleration measurement data, historical heart rate measurement data, and/or the like), historical event log data, historical geolocation data, and other historical or contextual data are correlated to or predictive of the occurrence of a particular event, activity, or metric for a particular patient, and then determines a corresponding equation, function, or model for calculating the value of the parameter of interest based on that set of input variables. Thus, the model is capable of characterizing or mapping a particular combination of one or more of the current (or recent) sensor glucose measurement data, auxiliary measurement data, delivery data, geographic location, patient behavior or activities, and the like to a value representative of the current probability or likelihood of a particular event or activity or a current value for a parameter of interest. It should be noted that since each patient's physiological response may vary from the rest of the population, the subset of input variables that are predictive of or correlative for a particular patient may vary from other users. Additionally, the relative weightings applied to the respective variables of that predictive subset may also vary from other patients who may have common predictive subsets, based on differing correlations between a particular input variable and the historical data for that particular patient. It should be noted that any number of different machine learning techniques may be utilized by the remote device 514 to determine what input variables are predictive for a current patient of interest, such as, for example, artificial neural networks, genetic programming, support vector machines, Bayesian networks, probabilistic machine learning models, or other Bayesian techniques, fuzzy logic, heuristically derived combinations, or the like.

As described in United States Patent Application Publication No. 2018/0174675, which is incorporated by reference herein in its entirety, a so-called personalized closed-loop (PCL) system may utilize artificial intelligence, machine learning, statistical models, and the like, to discover correlations between data collected at or around various events and the patient's individual habits, behaviors, physiological responses, and the like. In this regard, human beings are creatures of habit and as such many individuals have strong recurring event patterns (e.g., meal patterns, sleep patterns, exercise patterns, and/or the like). Thus, a PCL system can leverage predictive algorithms to exploit such patterns to lower the burden on the patients while maintaining safe glucose levels. For purposes of explanation, the subject matter may be described herein primarily in the context of meal prediction; however, it should be noted that the subject matter described herein is not limited to meal events may be implemented in an equivalent manner in connection with sleep, exercise, or any other type of event.

In order to identify future events, the PCL system passively collects data (e.g., without user input) from the patient's mobile devices (e.g., the CCD 106, computing device 108, client device 506, etc.), medical devices (e.g., infusion devices 102, 202, CGMs or other monitoring or sensing devices 104, 204, 206, 208, 504, etc.), or other devices associated with the patient (e.g., other wearable devices). The resulting data set may include various data points that can be used as cues to future meal or lifestyle events, such as, for example, glucose levels, historical meal info, acceleration or motion sensor data, geographic location data, network connectivity data, and the like. Thus, instead of the patient having to carry the mental burden of tracking an event such as a meal (e.g., counting carbohydrates, determine boluses, etc.), the PCL system may make predictions and provide corresponding notifications to the patient (e.g., notifying the patient when a meal is predicted such that the patient will only need to approve a bolus or modify it to his or her individual needs). In some examples, the PCL system may ask the patient to confirm a predicted event.

While some existing machine learning or other artificial intelligence techniques are capable of performing well when making predictions from complex data sets, such “black box”-type models suffer from a lack of explainability, which can be a limiting factor when used with medical devices or in other regulated environments where regulators, healthcare providers, patients, and the like expect a certain level of understanding of the model. On the other hand, traditional models like decision trees may be explainable but fail to achieve adequate performance in a complex environment or with respect to complex data sets. Accordingly, the subject matter described herein utilizes association mining or association harvesting to derive personalized machine learning models that achieve the desired level of prediction performance while also achieving a desired level of explainability by harvesting associations between taxonomies of various events for which data is collected for a given patient. By mapping meals to a particular meal ontology, improved predictions may be made by leveraging statistical correlations between different levels in the ontology and other preceding events. Additionally, the explainability may accelerate the regulatory path or certification of medical devices, facilitate quality assurance, and provide insights for patients, physicians, and other healthcare providers.

For example, in contrast to “black box” type models, the ontological modeling described herein allows for the sequence of contextual events or patient states that drive a particular recommendation, prediction, or action can be output or otherwise made visible to a patient, developer, regulator, or other user in a so-called “white box” manner. This transparency can improve quality assurance and confidence in the ontological predictions by improving human understanding of the algorithms and the underlying reasoning behind the predictions, while also improving the patient, developer, regulator, or other user identifying potential anomalies to be remediated. For example, a graphical user interface (GUI) display may allow for the nature of a “white box” ontological prediction model to be communicated to a user in a way that allows the user to comprehend, and potentially modify, the reasoning underlying the model used to generate predictions, recommendations, and the like.

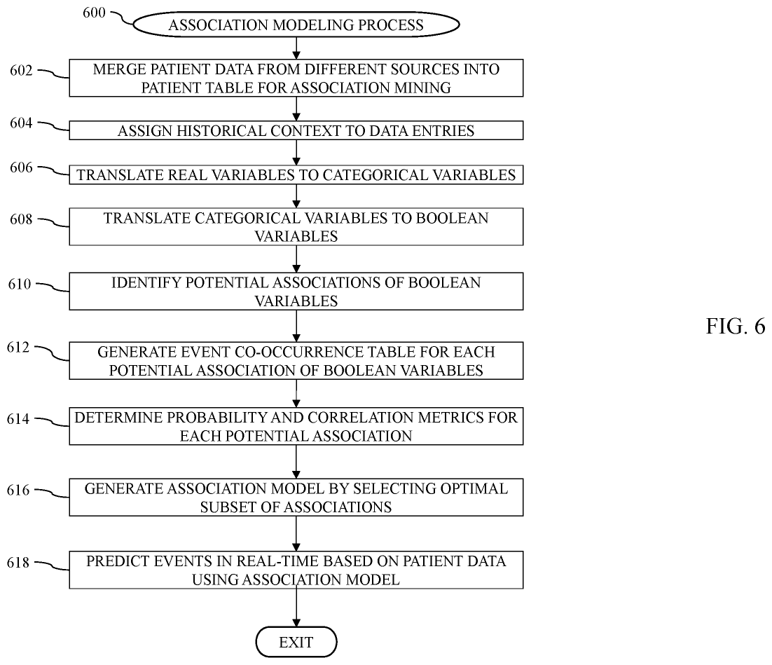

FIG. 6 depicts an exemplary association modeling process 600 suitable for implementation in a patient monitoring system, infusion system, or the like to predict occurrence of events and provide corresponding notifications, recommendations, or other responsive actions based on associated patient behaviors. The various tasks performed in connection with the association modeling process 600 may be performed by hardware, firmware, software executed by processing circuitry, or any combination thereof. For illustrative purposes, the following description refers to elements mentioned above in connection with FIGS. 1-5. In practice, portions of the association modeling process 600 may be performed by different elements of a system, such as, for example, an infusion device 102, 202, a sensing device 104, 204, 504, or other medical device 502, a client computing device 106, 506, a remote computing device 108, 514, and/or a pump control system 220, 300. It should be appreciated that the association modeling process 600 may include any number of additional or alternative tasks, the tasks need not be performed in the illustrated order and/or the tasks may be performed concurrently, and/or the association modeling process 600 may be incorporated into a more comprehensive procedure or process having additional functionality not described in detail herein. Moreover, one or more of the tasks shown and described in the context of FIG. 6 could be omitted from a practical embodiment of the association modeling process 600 as long as the intended overall functionality remains intact.

The association modeling process 600 begins by merging or otherwise formatting patient data obtained from different data sources into a single table (task 602). In this regard, an individual patient's meal data or other event log data obtained via the client application 508 is combined with the patient's glucose measurement data obtained via a sensing arrangement 104, 204, 504, bolus data or other insulin delivery obtained via an infusion device 102, 202, 502, and other contextual data obtained via other sensing arrangements 206, 208 (e.g., GPS data or other location data, motion data, heartrate data, and/or the like) or the patient's associated devices (e.g., network connectivity data, charger connectivity data, screen time data, and/or the like). In one or more embodiments, the remote server 514 utilizes one or more unique patient identifiers associated with the patient to be modeled to query the database 516 (and various tables or data sets maintained therein) for the various data entries associated with the patient. After collecting the patient's recent and historical data, the remote server 514 transforms or otherwise combines the different data entries into a table for further analysis.

In exemplary embodiments, the various pieces of patient data are merged into a single table that is indexed by date and time. In an exemplary embodiment, the table is configured to provide 5-minute intervals between consecutive rows in the table, where the rows are independent and contain values for each of the fields of patient data defining the operational context for the respective row's assigned date and time. For example, data sets from different sources may be merged with an outer join by date and time, resulting in a table containing all the different pieces of data ordered sequentially by date and time. Thereafter, the table may be resampled by date and time to provide 5-minute (or any other period of time) intervals between every pair of consecutive rows. In this regard, rows that are within 5 minutes (or other particular period of time) of one another may be combined into a single row associated with a common date and timestamp. Additional rows may also be inserted or otherwise created within the table to fill gaps within the data sets.

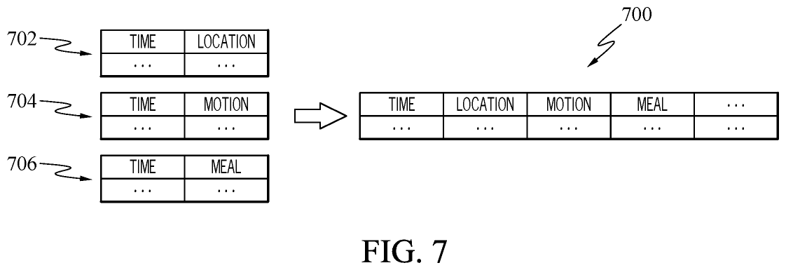

For example, FIG. 7 depicts an example of a merged row 700 of patient data that contains fields (or columns) of values for different patient data variables obtained from different data sets. In this regard, the merged row 700 is created by combining data entries 702, 704, 706 from different data sources that are timestamped with a common date and respective times that are at or within a threshold amount of time of the timestamp associated with the merged row 700. In the illustrated embodiment, a first data entry 702 containing geographic location data associated with the patient, which may be obtained via a GPS receiver integrated with the patient's client device 506, medical device 502, or other sensing arrangement 206 associated with the patient, is merged with a second data entry 704 containing acceleration or motion sensing data (e.g., obtained via an acceleration sensing arrangement 208) and a third data entry 706 including meal data (e.g., a meal indicator provided via the client application 508). In this regard, the different data entries 702, 704, 706 are transformed into a single data entry or row associated with the same data and substantially the same timestamp, thereby establishing logical and temporal associations between the different pieces of data from different data sets.

Referring again to FIG. 6, in exemplary embodiments, the association modeling process 600 continues by assigning historical contextual data to different data entries (task 604). In one or more exemplary embodiments, the association modeling process 600 generates one or more fields of metadata to be associated with the various entries in the patient data table. For example, the remote device 514 may calculate or otherwise determine temporal metadata variables to be associated with individual entries, such as, for example, an amount of elapsed time since the most recent preceding event of a given event type, an amount of elapsed time in the same geographic location, an amount of elapsed time since the most recent preceding meal bolus or other insulin delivery, and/or the like. In exemplary embodiments, the association modeling process 600 backfills any gaps or missing values within fields or columns of data entries using the preceding and/or following row in the patient table. In this regard, fields of the table may have missing values in some of the rows, either due to a data for a certain variable not being available at a certain time (e.g. location, network connectivity, etc.) or because rows were created to fill gaps in time within the data set (e.g., to provide entries at 5-minute increments). The missing field values may be filled according to field-specific logic.

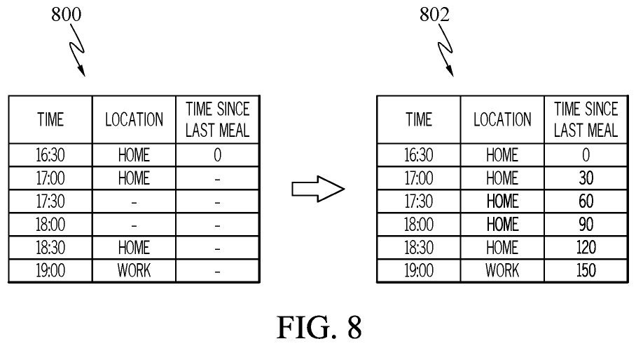

After filling gaps in the patient table, the association modeling process 600 calculates or otherwise determines values for metadata fields to be associated with the respective entries in the table. For example, FIG. 8 depicts an initial state 800 of a subset of a patient data table that depicts a location field having gaps across rows of the patient data table, along with a time since last meal metadata field that is initially populated with a value of 0 for an entry associated with a meal indicator. The association modeling process 600 updates the patient data resulting in an updated state 802 that eliminates the gaps in the location data field by applying logic to fill intermediary entries that reside between entries having the same location data field value with that location data field value (e.g., “Home”). Additionally, the association modeling process 600 calculates or otherwise determines the time since last meal metadata field values for the data entries that are not associated with a meal indicator based on the differences between the timestamp associated with the respective row and the timestamp associated with the row associated with the most recent meal.

In exemplary embodiments, after filling gaps in patient data fields and assigning metadata to different data entries, the association modeling process 600 generates or otherwise creates a second table for maintaining or otherwise tracking the historical context for each data entry. In this regard, the historical context table has the same date and time index entries, with each row containing historical contextual information for different fields of patient data. For example, in the initial patient table, the location data contained in the row may be limited to the current location at the particular date and time associated with the respective row, while in the historical context table, the location data contained in the row includes the current location at the particular date and time associated with the respective row, the amount of elapsed time in that location, the patient's preceding or previous location, the amount of elapsed time in that previous location, the patient's next preceding location, the amount of elapsed time in that location, and so on as desired. As another example, in the initial patient table, the network connectivity data contained in the row may be limited to a Boolean value for whether or not network connectivity existed at the particular date and time associated with the respective row, while in the historical context table, the network connectivity data may include the Boolean value for whether or not network connectivity existed at the particular date and time associated with the respective row as well as the elapsed time in that particular state (e.g., wi-fi connectivity for 2 hours).

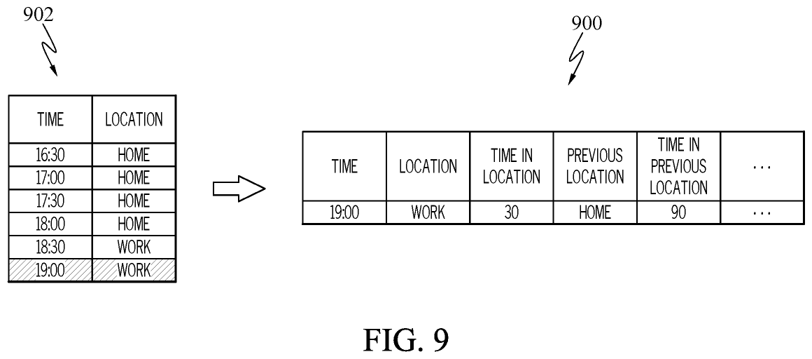

FIG. 9 depicts an exemplary subset of an entry 900 in a historical context table that may be generated for the patient's location based on the location data field values for a subset 902 of the initial patient table. For example, for the historical context table entry 900 assigned to the 19:00 timestamp, the association modeling process 600 may populate the entry with the current location field value (e.g., “Work”) from the corresponding 19:00 timestamped entry in the initial table. Based on the location data field values preceding the 19:00 timestamped entry in the initial table, the association modeling process 600 calculates or otherwise determines the elapsed time in the current location as of the timestamped time of 19:00 (e.g., 30 minutes), the location preceding the current location (e.g., “Home”), the total elapsed time in the preceding location (e.g., 90 minutes), and so on, and then populates the historical context table entry 900 for the 19:00 timestamp with those historical location contextual data values derived from the initial table.

Referring again to FIG. 6, after merging data sets into a patient table and generating a corresponding historical context table, the association modeling process 600 continues by converting, transforming, or otherwise translating real patient data variable values into categorical variable values (task 606). In this regard, real numbers that represent the value or state of a particular data field are mapped or otherwise converted into a categorical value that represents one of a finite, predefined set of possible values for that data field. In this manner, the real data values are classified or otherwise categorized into logically different categorical values. In exemplary embodiments, for each real-valued patient data variable in the patient data table, a hierarchical clustering is performed to define different states or classifications for that patient data variable. Thereafter, the real values for that patient data variable may be transformed into a corresponding categorical value (or cluster identifier). Thus, the real data values are effectively encoded with the appropriate categorization or classification in lieu of the real value. As a result, substantially similar values for a particular patient data field may be treated similarly, even though the underlying real values are not identical. In one or more embodiments, the clustering is performed based only on rows that include meals (or the other event to be modeled), so that the clustering will be oriented around the values encountered in connection with meals.

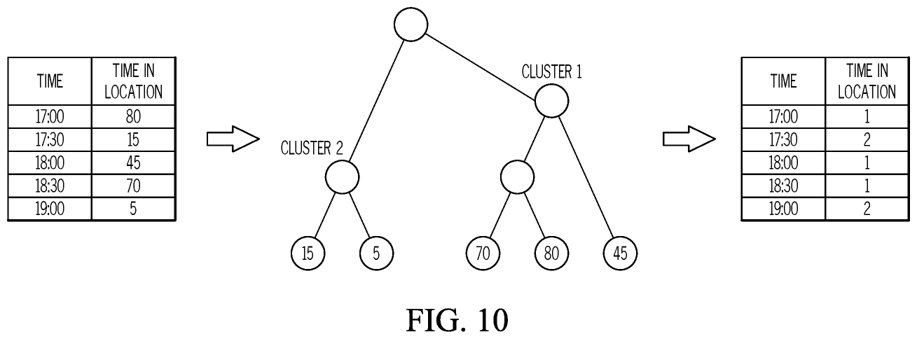

FIG. 10 depicts an exemplary hierarchical clustering and transformation of the time in location patient data variable value from real values representing the elapsed time in the current location at the time of a meal (or other event) into a corresponding cluster or category for the elapsed time at the meal location at the time of the meal. In this regard, any number of different clustering techniques may be performed to divide the real data values based on their relative similarity or dissimilarity into a desired number of clusters, and the subject matter described herein is not intended to be limited to any particular clustering algorithm or technique. In the illustrated embodiment depicted in FIG. 10, the time in location values of 45 minutes, 70 minutes and 80 minutes are assigned to a first cluster (e.g., cluster ID=1) and the time in location values of 5 minutes and 15 minutes are assigned to a second cluster (e.g., cluster ID=2). Thereafter, the association modeling process 600 updates or otherwise overwrites the original real values for the elapsed time in the current location with the categorical values in lieu of the original real values.

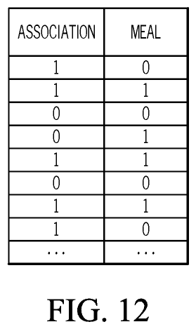

Referring again to FIG. 6, after translating real patient data variable values into categorical values, the association modeling process 600 continues by converting, transforming, or otherwise translating categorical patient data variable values into Boolean variable values (task 608). In this regard, each patient data variable field may be converted into a number of different fields corresponding to the different categorizations or clusters associated with that patient data variable field. The resulting columns of a data entry may then be populated with Boolean values of ‘1’ or ‘0’ to identify what the respective categorization or classification of the patient data variable field was for that respective timestamped entry. In this regard, the column corresponding to that entry's assigned category may be set to the Boolean value of ‘1’ to indicate classification into that respective category associated with that field, while the other columns for that patient data variable field are set to the Boolean value of ‘0’ to indicate the patient data variable field for the respective entry are not assigned to those categories. For example, as depicted in FIG. 12, a timestamped entry associated with a time of 16:30 and an associated location of “Home” may be transformed into a corresponding timestamped entry with a Boolean value for a “Location=Home” field set to the value of ‘1’ and the Boolean value for a “Location=Work” field set to the value of ‘0’, and so on. Similarly, the 17:00 timestamped entry with an associated location of “Home” may be transformed into a corresponding timestamped entry with a Boolean value for a “Location=Home” field set to the value of ‘1’ and the Boolean value for a “Location=Work” field set to the value of ‘0’, while the 17:30 timestamped entry with an associated location of “Work” may be transformed into a corresponding timestamped entry with a Boolean value for a “Location=Home” field set to the value of ‘0’ and the Boolean value for a “Location=Work” field set to the value of ‘1’, and so on.

Referring again to FIG. 6, after transforming the patient data variable values to Boolean values, the association modeling process 600 continues by identifying or otherwise determining potential contextual state associations or combinations of Boolean values across the different patient data variables, and for each potential contextual state association, generates or otherwise creates an event co-occurrence table for each potential contextual state association and calculates or otherwise determines probability and correlation metrics for the potential event of interest for the potential contextual state association based on the co-occurrence table (tasks 610, 612, 614). A contextual state association generally represents a combination of concurrent state values for two or more patient data fields, which may or may not be correlative to or predictive of the occurrence of an event. For example, a contextual state association of a location field having a categorical state value corresponding to the patient being at work (e.g., the “location=work” field having a Boolean value of ‘1’) and a time in location field having a categorical state value corresponding to the patient being at the current location for around 3 hours (e.g., a Boolean value of ‘1’ for the “time in location=˜3 hours” field) corresponds to situations where the patient has been at work for around 3 hours. Thus, contextual state associations represent aspects of the patient's routine behavior (e.g., getting up in the morning and eating breakfast, going out for lunch during work, etc.). In practice, the association modeling process 600 attempts to generate as many potential different combinations of concurrent state values for two or more patient data fields as possible that are also associated with one or more instances of a meal (or other event to be modeled). Here, it should be noted that while the contextual state associations may be described here in the context of textual labels or characterizations, in practice, the associations may be defined by combinations of numerical identifiers representing different patient data field state combinations.

For each potential contextual state association, the patient's data table is analyzed to identify, for each row, whether the row includes a meal indicator or is otherwise associated with a meal, or whether the row includes the contextual state association being analyzed. A co-occurrence table is generated that includes a subset of entries from the patient's data table where either the contextual state association of interest was present (or activated), a meal indication was present, or neither the contextual state association nor the meal were present. For example, as shown in FIG. 12, a first entry in the co-occurrence table may be created to correspond to an entry in the patient's data table having a Boolean value of ‘1’ for each of the “location=work” field and “time in location=˜3 hours” fields but a Boolean value of ‘0’ for a meal indicator field. The second entry corresponds to another entry in the patient's data table having a Boolean value of ‘1’ for each of the “location=work” field and “time in location=˜3 hours” fields and a Boolean value of ‘1’ for a meal indicator field, the third entry corresponds to another entry in the patient's data table having a Boolean value of ‘0’ for each of the “location=work” field and “time in location=˜3 hours” fields and a Boolean value of ‘0’ for a meal indicator field.

In one or more exemplary embodiments, to calculate the probability and correlation metrics, a contingency table is created based on the co-occurrence table that tracks the co-occurrence of the presence or absence of the identified contextual state association and the meal (or other event being modeled), that is, the number of entries corresponding to each potential combination of contextual state association presence or absence and meal presence or absence (e.g., the total number of instances of both the contextual state association and meal being present, the total number of instances of just the contextual state association being present, the total number of instances of just the meal indicator being present, and the total number of instances where neither the meal nor the contextual state association were present). From the contingency table, the probability of a meal given the contextual state association (or meal prevalence) is calculated as a ratio of the total number of instances where both the contextual state association and meal were present to the sum of the total number of instances where both the contextual state association and meal were present and the total number of instances of where the contextual state association was present without a meal indicator. In this regard, the meal prevalence may be calculated as a function of the total number of instances where both the contextual state association and meal were present and the total number of instances of where the contextual state association is present without the meal indicator being present.

In exemplary embodiments, to calculate the correlation metric representing the correlation significance associated with the contextual state association, a binomial discrete probability distribution is determined using the distribution's survival function (complement of the cumulative distribution function). The binomial discrete probability distribution represents the discrete probability of the number of successes in a particular number of independent experiments with a particular probability of success, and the survival function represents the probability that, for a certain event, the outcome variable will be greater than some value. In this regard, the correlation metric may be determined using as a function of the total number of instances of where the meal indicator is present without the contextual state association being present, the total number of instances where neither the contextual state association or meal indicator was present, and the total number of entries in the co-occurrence and contingency tables. The correlation significance generally represents the probability of observing a co-occurrence at least as strong as the observed co-occurrence randomly (e.g., if there is no correlation). In this regard, smaller values for the correlation significance metrics indicates a stronger correlation significance.

Still referring to FIG. 6, in exemplary embodiments, the association modeling process 600 generates or otherwise determines the association model for predicting the event of interest by selecting or otherwise identifying an optimal subset of the potential contextual state associations for the patient (task 616). In exemplary embodiments, a genetic algorithm is utilized to iteratively generate a subset of contextual state associations in accordance with optimization criteria. In this regard, a multi-objective genetic algorithm is utilized to optimize the meal probability (or meal prevalence) and the correlation significance for the subset of contextual state associations. The multi-objective genetic algorithm generates a pool of contextual state associations from which a prediction model for the meal (or event) is derived. To iteratively arrive at an optimal subset of contextual state associations, the multi-objective genetic algorithm generates an initial random pool of potential subsets of contextual state associations, ranks the potential subsets of contextual state associations according to the optimization criteria, sorts the potential subsets of contextual state associations according to their rank, and generates another pool of potential subsets of contextual state associations based on the highest ranked subset of contextual state associations that also includes new potential subsets of contextual state associations resulting from combining subsets from the initial pool and/or new potential subsets of contextual state associations resulting from modifying subsets from the initial pool, and then iteratively repeats the steps of ranking, sorting, and generating additional pools of potential subsets of contextual state associations until meeting a stopping criteria (e.g., when the highest ranked subset is unchanged or otherwise maintained the same score across a threshold number of successive iterations). For the multi-objective genetic algorithm, the association modeling process 600 ranks the potential subsets of contextual state associations in accordance with their respective meal probability metrics and correlation significance metrics and attempts to find the pareto front.

In one or more exemplary embodiments, the aim of the association selection stage is to select the subset of contextual state associations that has a relatively high general correlation significance as well as relatively high precision and recall. In this regard, in some embodiments, these metrics may be combined into one variable to be optimized. The combination may be done by first calculating a combined score based on the precision and recall metrics, and then multiplying the result by a damping coefficient which is calculated from the general correlation significance. The precision metric represents the proportion of true positives (e.g., actual occurrence of a meal for which the model predicted a meal) with respect to the total number of predicted positives with respect to a given association model (or subset of contextual state associations), while the recall metric represents the proportion of true positives with respect to the total number of actual positives. For each association subset, the precision and recall metrics may be calculated by applying the association subset to a training subset of the patient's data to obtain a predicted number of meal indications by the association subset with respect to the total number of meal indications contained in the training subset. In this regard, the association subset is used to predict mealtime by predicting a meal whenever the current patient context matches a contextual state association which belongs to the subset and the current time associated with the current patient context is at or within a threshold time period of a time period associated with the contextual state association. For example, if the association subset includes the contextual state association of “location=home” and “time in location=˜2 hours,” and the threshold time period is set at 30 minutes, then a meal is predicted for each instance within the training data where the location data indicates the patient is at home and has been at home for between 1 hour and 45 minutes and 2 hours and 15 minutes (e.g., a 30-minute window centered about a duration of 2 hours). Thus, if a patient routinely eats a meal about 2 hours after arriving home, a meal may be predicted whenever that scenario is exhibited within the training subset of patient data.

In exemplary embodiments, the precision and recall metrics are combined into a single metric of classification success called an F-score. The F-score may be calculated as a function of a precision metric, a recall metric, and a parameter that represents the desired importance ratio between precision and recall. In an exemplary embodiment, the parameter β is chosen to be a value that prioritizes precision over recall for meal predictions. For each association subset, the precision, recall, and corresponding F-score are calculated and then weighted by the general correlation significance for the association subset as a function of the correlation metric or correlation significance for the respective contextual state associations in the subset using a regularization parameter. In exemplary embodiments, the tasks 610, 612, 614 and 616 are iteratively performed until arriving at a subset of contextual state associations that optimizes the weighted F-score. The resulting subset of contextual state associations functions as the predictive association model for the patient, where each respective contextual state association of the subset represents a patient behavior or habit that is predictive of a meal (or other event occurrence). In some embodiments, the resulting predictive association model is applied to a testing subset of the patient's data to confirm the predictive association model satisfies one or more applicable performance thresholds (e.g., a threshold level of precision, a threshold level of recall, a threshold F-score, and/or the like) before deploying the predictive association model to one or more of the patient's devices for real-time use of the predictive association model.

Still referring to FIG. 6 and with reference to FIG. 5, after deriving a predictive association model for the patient, the association modeling process 600 continues by predicting occurrence of events in real-time using the predictive association model (task 618). In this regard, each time anew piece of data is available from one or more data sources within the patient monitoring system 500, the predictive association model may be applied to the recent data characterizing the patient's current context or situation to identify or otherwise determine whether the patient's current behavior corresponds to a contextual state association within the subset of contextual state associations that make up the predictive association model. For example, the remote server 514 may analyze the historical data associated with the patient in the database 516 to derive the patient's specific predictive association model, which may then be pushed, downloaded, or otherwise transmitted to one of the patient's associated devices 502, 504, 506. At the device 502, 504, 506, the predictive association model may be applied in real-time in response to new data samples to detect or otherwise identify when the patient's current behavior matches one of the contextual state associations of the model. When the patient's current behavior or contextual state corresponds to one or more contextual state associations in the patient's predictive association model, the respective device 502, 504, 506 may generate or otherwise provide a user notification indicative of the predicted event or initiate some other action in anticipation of or in response to the predicted event.

For example, in one or more embodiments, the client application 508 may continually analyze the patient's recent location data, glucose measurement data, motion data, and/or the like to detect or otherwise identify when the patient's current contextual state matches a contextual state association of the patient's predictive association model, and in response to detecting a match, the client application 508 may automatically generate or otherwise provide a notification to the patient, automatically provide a bolus wizard graphical user interface (GUI) display on the client device 506, or otherwise indicate a meal was predicted to remind the patient to administer a meal bolus if necessary. As described in U.S. Patent Application Pub. No. 2018/0174675, in some embodiments, in response to a predicted meal, one or more parameters of the closed-loop control system 400 may be prospectively adjusted to account for an anticipated meal. In other embodiments, meal sizes or carbohydrate amounts, meal content, or other attributes or aspects of the meal may be predicted and utilized to assist or facilitate the patient administering a meal bolus. Thus, instead of the patient having to carry burdens of meal tracking and carb counting, the patient may instead be automatically notified when a meal is believed to be likely such that the patient may only need to confirm the meal, approve a proposed bolus amount, and/or the like. Reducing the amount of user interaction and corresponding graphical user interface (GUI) displays and related processing may also help prolong the battery life of the patient's associated device(s) which would otherwise be utilized for manually tracking or logging meals, performing carb counting, configuring boluses, and/or the like. Moreover, in some embodiments, an infusion device may be commanded or otherwise configured to automatically and autonomously respond to predicted meal, for example, by automatically delivering the proposed bolus amount to the patient. It should be noted the subject matter described herein is not intended to be limited to any particular action to be initiated in response to detecting presence of a contextual state association that is predictive of occurrence of a particular event, nor is the subject matter described herein intended to be limited to any particular device or component of a patient monitoring system which may use a patient's predictive association model to generate such predictions and initiate such actions. In this regard, while the subject matter is described herein primarily in the context of predicting the occurrence of a meal, it could be implemented in an equivalent manner to predict an amount of carbohydrates to be consumed, the meal content, or other meal attributes using the association modeling process 600. Likewise, the association modeling process 600 could be utilized to predict other events likely to impact the patient's glycemic condition (e.g., exercise or the like) and initiate corresponding actions to account for the predicted event.

In exemplary embodiments, the predictive association model is utilized in accordance with other logic that eliminates or otherwise prevents redundant notifications or actions responsive to an event. For example, if the patient has already announced a meal, delivered a meal bolus, and/or the like, use of the predictive association model may be paused or otherwise deactivated for a period of time until the patient's current contextual state changes to one in which the predictive association model does not predict a meal (e.g., when the patient's subsequent contextual state or behavior corresponds to an association of behaviors that does not belong to the subset that makes up the patient's predictive association model).

In one or more embodiments, the association modeling process 600 is initiated or otherwise performed on a periodic basis (e.g., daily, weekly, or the like) by the remote server 514 to dynamically update the patient's predictive association model to reflect changes in the patient's behavior or habits over time and reflect more recent patient data. In this regard, an older percentage of the patient's historical data may be utilized as the training subset of patient data, while the remaining more recent percentage of the patient's historical data may be utilized as the testing subset of patient data to verify or confirm the performance of the predictive association model. In this regard, when a more recently developed predictive association model does not meet or exceed applicable performance thresholds or otherwise does not perform as well as the preceding instance of the patient's predictive association model, the existing predictive association model may be maintained until a better performing model is developed.

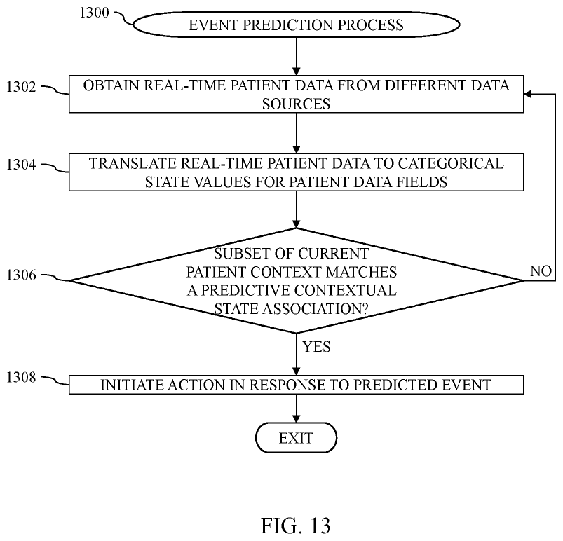

FIG. 13 depicts an exemplary embodiment of an event prediction process 1300 for predicting occurrence of an event using a predictive association model derived as part of the association modeling process 600. In this regard, the event prediction process 1300 may be implemented or otherwise performed by one of a patient's associated devices 502, 504, 506 after obtaining a predictive association model derived from the patient's historical data from the remote server 514. The various tasks performed in connection with the event prediction process 1300 may be performed by hardware, firmware, software executed by processing circuitry, or any combination thereof. For illustrative purposes, the following description refers to elements mentioned above in connection with FIGS. 1-5. It should be appreciated that the event prediction process 1300 may include any number of additional or alternative tasks, the tasks need not be performed in the illustrated order and/or the tasks may be performed concurrently, and/or the event prediction process 1300 may be incorporated into a more comprehensive procedure or process having additional functionality not described in detail herein. Moreover, one or more of the tasks shown and described in the context of FIG. 13 could be omitted from a practical embodiment of the event prediction process 1300 as long as the intended overall functionality remains intact.

In exemplary embodiments, the event prediction process 1300 initializes or otherwise begins by receiving or otherwise obtaining real-time data associated with the patient from any number of potential data sources and identifying or otherwise determining current categorical state values for the patient for a plurality of different patient data fields (tasks 1302, 1304). For example, when the event prediction process 1300 is being implemented or otherwise performed at the client device 506 (e.g., by client application 508), the client device 506 may periodically receive updated sensed glucose measurement data from a sensing arrangement 104, 204, 504 associated with the patient, insulin delivery data from an infusion device 102, 202, 502 associated with the patient, acceleration or motion measurement data from an acceleration sensing arrangement 208 associated with the patient, and/or other measurement data obtained via other sensing arrangements 206 associated with the patient (e.g., location data, and/or the like). The client device 506 may also locally obtain data associated with the patient in real-time, such as location data (e.g., via a GPS receiver of the client device 506), network connectivity data, patient activity data (e.g., screen time data) and/or the like. In a similar manner as described above in the context of FIG. 6, the event prediction process 1300 converts, translates, or otherwise transforms the real-time data into categorical state values for different patient data fields. For example, the client device 506 may create or otherwise maintain, at the client device 506, a table that merges, combines, or otherwise associates temporally associated pieces of data from different data sources, which, in turn, may then be translated into categorical state values that define the current patient context in a similar manner as described above (e.g., tasks 606, 608).

Still referring to FIG. 13, the event prediction process 1300 continually analyzes the current categorical state values for the patient data fields that define the current contextual state of the patient to detect or otherwise identify when a subset of the current contextual state of the patient matches a contextual state association assigned to the patient's predictive association model (task 1306). In this regard, when the current contextual state of the patient encompasses, overlaps, or otherwise includes categorical state values for two or more fields that match those associated with one of the predictive contextual state associations for the patient, the event prediction process 1300 predicts the probable or likely occurrence of the event associated with the predictive association model. In response to predicting occurrence of the event, the event prediction process 1300 initiates or otherwise performs one or more actions that are influenced by or otherwise responsive to the predicted event (task 1308). For example, when a meal is predicted, the client application 508 may automatically generate or otherwise provide a user notification that a meal is predicted or expected, for example, by automatically generating a bolus wizard GUI display or automatically displaying or otherwise presenting a meal bolus recommendation at the client device 506. The patient may then interact with the client application 508 to confirm the occurrence of a meal, confirm, modify or adjust a bolus dosage of insulin to be delivered, or otherwise take a desired action with respect to the meal. In yet other embodiments, the client application 508 and/or client device 506 may automatically command, signal, or otherwise instruct an infusion device 102, 202, 502 associated with the patient to automatically adjust insulin delivery based on the predicted meal, for example, by prospectively adjusting the closed-loop control as described in U.S. Patent Application Pub. No. 2018/0174675. Again, it is noted that the subject matter described herein is not limited to any particular action or combination of actions to be initiated, nor is the subject matter limited to any particular type of event to be predicted.

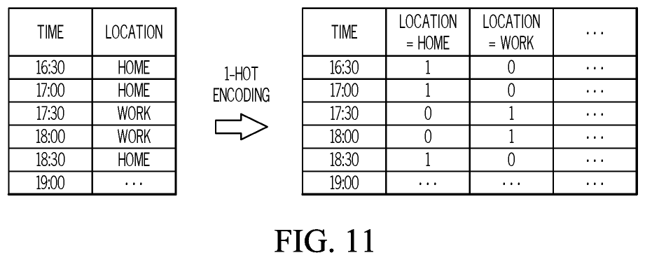

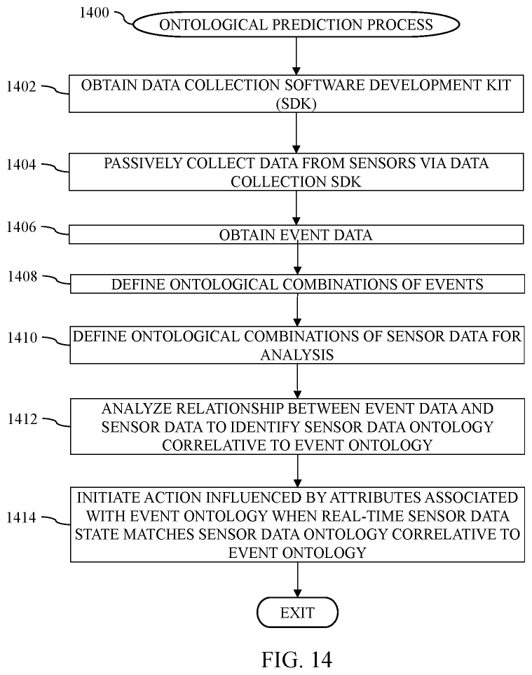

Referring to FIG. 14, with continued reference to FIGS. 1-13, many human beings are creatures of habit and as such many individuals exhibit strong recurring eating patterns. A personalized closed-loop system can leverage predictive algorithms to exploit this fact to lower the burden on the patients while maintaining safe glucose levels. This can be achieved by employing machine learning algorithms that study recurring patterns between a patient's eating habits and other parameters (as described in greater detail below) that can be used as predictors of such habits and thus identify future meals before they take place.

In order to identify future events, the personalized closed-loop system passively collects a rich dataset from the patient's smartphone, the patient's medical devices, and any other wearable devices. The resulting data set includes various data points that can be used as cues to future meal or lifestyle events, and include glucose levels, historical meal info, motion sensors from the phone, geolocation (e.g., using the smartphone's GPS), sleep events (including duration, sleep time and wakeup times), phone light sensors, and network connectivity information (e.g., Wi-Fi connectivity state and the like). In this sense, the personalized closed-loop system treats the patient's devices as a bed of continuous sensors that may provide further insight into a patient's behavioral patterns.

When a patient onboards the system for the first time, an application associated with the personalized closed-loop system may request system permissions to access sensors for the data points described above, most of which could be collected passively without the patient manually logging data. During the first days of usage the system is in its learning phase, where each interaction of the patient (or the lack thereof) is training the system on the patient's individual eating and lifestyle patterns. Artificial intelligence, machine learning and statistical models discover correlations between the various sensor data state combinations and/or sequences that were collected, and the patient's eating habits, exercise habits, or other lifestyle habits. The learning algorithms may be regularly and/or continually trained to identify behavioral patterns, and once confidence levels reach a sufficient level, the system will gradually start performing actions in attempts to lower the patient's burden. Thus, instead of the patient having to carry the mental burden of tracking meals and counting carbohydrates, the personalized closed-loop system can notify the patient before a meal is very likely to be eaten and/or automatically configure or initiate prospective remedial actions, and the patient may only need to approve a bolus or modify it to his or her individual needs or desires.

For example, consider a patient that eats lunch every day at one of a handful of restaurants nearby their workplace. As the patient's location changes to one of the restaurants, the application will identify the change of location, the time of day, the fact that it is a workday and the historical data about the meals that were previously eaten in that restaurant. With all this information, the algorithms may identify the new circumstances to be a likely scenario for a new meal taking place within the next minutes and notify the patient that this event was detected. The patient can then decide whether to activate the bolus, not to activate it, or even modify the carb amount if necessary.

In one example, a meal timing prediction model is utilized to predict the occurrence of future meals before they take place, and a meal content prediction model predicts the probable amount of carbohydrates that will be eaten in a meal. The meal content prediction model may operate under the assumption that a meal is taking place and only be activated once a determination is made that the user is about to eat a meal. Both models may receive as inputs the historical meal data of the user (eating times, carbohydrate amounts and meal content if available), glucose measurement data, location data, motion sensor data, wearable data like sleep and activity data (if available), light sensors and Wi-Fi network connectivity data. The output of the meal timing prediction model is a confidence level that a meal will occur soon for various time windows (equivalent to a probability) for a given set of inputs collected. The algorithm output provides the confidence in a meal taking place in the next 15 minutes, the next 30 minutes, the next 45 minutes, etc. Since a smaller window is a subset of the longer time windows the confidence level will only increase on wider time spans (e.g. the probability of a meal to be eaten in the next 15 minutes is smaller than the probability of a meal to be eaten in the next 30 minutes). The output of the meal content prediction model will be an estimation of a range for the carbs that are expected to be consumed by the user, and perhaps also a list of individual food items together with their amounts and nutritional information. In one or more exemplary embodiments, a deep neural network is utilized for the meal prediction model, while an association harvesting model is utilized for the meal content prediction model. As described in greater detail below, an association harvesting model attempts to harvest associations between taxonomies of the various events collected for a given patient. Each meal consumed by a patient may be mapped to one or more meal or event ontology, for which statistical correlations may be determined between different levels in the ontology and other events that preceded it (e.g. breakfast that is eaten between 8 and 9 AM always includes coffee and a pastry, the latter is either croissant or a donut).

FIG. 14 depicts an exemplary embodiment of an ontological prediction process 1400 for meal or other event predictions, which, in some embodiments may utilize a predictive association model derived as part of the association modeling process 600. The various tasks performed in connection with the ontological prediction process 1400 may be performed by hardware, firmware, software executed by processing circuitry, or any combination thereof. For illustrative purposes, the following description refers to elements mentioned above in connection with FIGS. 1-5. It should be appreciated that the ontological prediction process 1400 may include any number of additional or alternative tasks, the tasks need not be performed in the illustrated order and/or the tasks may be performed concurrently, and/or the ontological prediction process 1400 may be incorporated into a more comprehensive procedure or process having additional functionality not described in detail herein. Moreover, one or more of the tasks shown and described in the context of FIG. 14 could be omitted from a practical embodiment of the ontological prediction process 1400 as long as the intended overall functionality remains intact.

The illustrated ontological prediction process 1400 begins with at least one of a patient's associated devices downloading, installing, retrieving, or otherwise obtaining a software development kit (SDK) that supports passively collecting data and other tasks or processes related to the ontological mapping described herein (tasks 1402, 1404). For example, the SDK may be downloaded to a patient's phone (e.g., client device 506) from a remote device (e.g., remote server 514) and installed or otherwise implemented to support a client application (e.g., client application 508) that supports passive data collection. When a patient onboards the system for the first time, the SDK (or an application associated therewith) may request system permissions to access sensors for passive data collection. The SDK may then reside on the patient's phone and passively collect data without any interaction from the patient. In this regard, the SDK treats the phone and other devices communicatively coupled thereto (e.g., devices 502, 504) as a bed of sensors and listens for changes in the state or output data of the various sensors, including, for example, changes to the GPS coordinate location, Wi-Fi or other network connectivity states, optical or light sensors, accelerometers, gyroscopes, tilt sensors and/or other motion sensors, and/or the like that characterize the current operating environment or contextual state, in addition to glucose measurement data, insulin delivery data, and/or other data pertaining to the patient's physiological condition. In some embodiments, the passive data collection can also access a camera or photo gallery for analyzing and classifying photos, videos, or other image data available at or on a given device (e.g., to classify photos of food that were taken). The SDK encrypts the various streams of data collected and analyzed by the SDK and uploads the encrypted passively-collected data to the cloud (e.g., remote device 514) for further analysis. In this regard, the passively collected data includes data for the patient obtained from wearable devices (e.g., smart watches, trackers, pedometers, and/or the like) and/or associated medical devices in addition to data obtained locally at the patient's phone or client device where the SDK resides, such as, for example, glucose levels, acceleration or other motion data, geolocation, light sensor data, network connectivity and/or the like. This allows for the collection of thousands of data points on a daily basis without any interference with the lifestyle of the patient, and therefore, without any patient burden. For purposes of explanation, the passively-collected data obtained by the SDK and uploaded to the cloud may alternatively be referred to herein as sensor data.

The ontological prediction process 1400 also collects or otherwise obtains event data associated with the patient (task 1406). In this regard, the SDK or other application at the patient's phone may be configured to provide an event logging GUI or other feature or application that allows the patient to input or otherwise define meals, exercise, and/or other activities that the patient engages in, for example, as described in U.S. Patent Application Pub. No. 2018/0137938. The event log data is uploaded or otherwise provided to a remote device (e.g., remote server 514) for analysis with respect to the other passively collected data obtained by the SDK, as described in greater detail below.

Still referring to FIG. 14, the ontological prediction process 1400 continues by generating or otherwise defining ontological combinations of the event data for analysis (task 1408). In this regard, the remote device analyzes the event log data to identify different combinations or sequences of events for analysis that correspond to distinct patterns of patient behavior, such as, for example, sleep followed by exercise, sleep followed by a meal, exercise followed by a meal, exercise followed by sleep, and so on. Thus, different combinations or sequences of events (or event state associations) may be defined for analysis. Additionally, the different ontological event combinations may incorporate time or other temporal relationships between events, for example, exercise in the afternoon followed by a meal within one hour of exercise may be a distinct event ontological combination from exercise in the afternoon followed by a meal more than one hour after exercise or exercise in the morning followed by a meal within one hour after exercise. The ontological combinations may also be defined at varying levels of specificity or granularity, for example, by incorporating additional event attributes into the definition of a given event ontology, such as a particular type of exercise (e.g., running, walking, strength training, aerobic or anaerobic exercise, etc.) followed by a particular type of meal or meal content and/or a particular size of meal within a particular timeframe of the particular type of exercise. As described above, during the first days of usage, the system may enter learning phase, where each interaction of the patient (or the lack thereof) collected by the SDK is utilized to train the system on the patient's individual eating and lifestyle patterns.

Additionally, the ontological prediction process 1400 continues by generating or otherwise defining ontological combinations of the passively-collected sensor data for analysis (task 1410). In this regard, the remote device analyzes the uploaded sensor data that was passively-collected by the SDK to define different ontological combinations or sequences of sensor data for analysis that correspond to distinct patterns of patient behavior or behavioral states (e.g., contextual state associations), such as, for example, a particular combination or sequence of GPS locations and concurrent network connectivity states, glucose measurement values (or ranges thereof), infusion device mode settings (e.g., closed-loop mode, open-loop mode, suspended delivery, and/or the like). For example, the remote device may apply artificial intelligence, machine learning and statistical models to the passively-collected data to discover correlations between the various sensor data state combinations and/or sequences that were collected, and the patient's eating habits, exercise habits, or other lifestyle habits. Moreover, the remote device may periodically and/or continually retrain and/or update the learning algorithms to identify new or different behavioral patterns. Once confidence levels for a particular sensor data state combination and/or sequence reaches a sufficient level, the system will gradually start performing actions responsive to a subsequent occurrence of particular sensor data state combination and/or sequence in a manner that attempts to lower the patient's burden. It should be noted that similar to the event ontologies, different combinations or sequences of sensor data may incorporate time or other temporal relationships between different types of sensor data (e.g., a particular GPS location followed by a particular network connectivity state, etc.). The sensor ontologies may also be defined at different levels of specificity or granularity.

After defining different potential event ontologies and sensor ontologies, the ontological prediction process 1400 continues by analyzing the relationships between sensor ontologies and event ontologies to identify particular ontological combinations of sensor data that are predictive of or correlative to occurrence of a particular event ontology (task 1412). In this regard, the remote device may perform association mining to identify a given sensor ontology that is predictive of or correlative to a particular event ontology.

In exemplary embodiments, the sensor and event data are managed as time series data that are mapped to fixed time increments (e.g., 5 minute intervals) to achieve a database of phone-related contexts, lifestyle habits and meals or other events characterized by the data from the various phone sensors, the meal or other event attributes from the event log data, glucose measurement data, and insulin delivery data. The relationships between event and sensor ontologies defined or derived from the database of phone-related contexts and lifestyle habits are then analyzed to identify patterns in the life of a given patient, such as, for example, when meals are normally consumed, where meals are normally consumed, what is normally consumed at those particular times in locations, what events or other patient states occurred beforehand, when patients wake up, when patients go to sleep, and so on. In this regard, it should be noted that while people may be creatures of habits, many of the patterns that emerge in some habits are non-linear and non-trivial to identify. In order to address this, the event and sensor data are mapped into the various ontological combinations and patterns or correlations between a sensor ontological combination and an event ontological combination may be identified on different levels of the ontology. For example, a person may consume for breakfast coffee and either a donut or a croissant. It may be unclear for the data collected from the phone sensors whether this individual patient will consume a croissant or a donut at a given data, as it may depend on individual choice or a type of data that is not available in a non-passive way through the phone sensors. However, the food ontology tells us that croissants and donuts are both special cases of pastries. The algorithm can find that this pattern, at a higher level of the ontology, is stronger and more statistically significant than other patterns, and thus allows making a more accurate prediction of the probable meal content, the probable meal size, and/or other meal attributes.

In a similar way, other characteristics on various levels and data types, can be used to identify patterns, such as: the type of workouts, the time of workouts (on levels of minutes, hours, days, weekdays vs. weekends, etc.), the ingredients, nutritional or macronutrient content of food being consumed, the manner in which the food is processed, types of cuisines consumed, location (on the level of a specific venue, a specific area, a specific city, etc.), and so on.

After identifying sensor data ontologies that are predictive of a particular event ontology, the ontological prediction process 1400 proceeds by initiating one or more actions that are influenced by one or more attributes associated with a particular event ontology when real-time sensor data corresponds to a sensor data ontology that is correlative to or predictive of that event ontology (task 1414). In this regard, the SDK (or an application associated therewith) residing on one of the patient's devices continually monitors the sensor data that is being passively collected from different data sources in real-time to detect or otherwise identify when the combination or sequence of current and/or recent sensor data values matches or otherwise corresponds to a particular sensor data ontology, which, in turn, is correlative to, predictive of, or otherwise associated with a particular event ontology. When the current sensor data state or sequence corresponds to an ontological sensor data state that is correlative to an event ontology, the SDK predicts the occurrence of the event(s) associated with the event ontology.

For example, as described above, based on the patient's location at one of a handful of restaurants nearby their workplace, the time of day, the fact that it is a workday and the historical data about the meals that were previously eaten in that restaurant, the SDK may predict a new meal taking place within the next few minutes having estimated or predicted nutritional characteristics based on previous meals at that location and time of day (e.g., mean historical carbohydrate amount, etc.), and then notify the patient of the predicted meal and its predicted nutritional characteristics and a corresponding recommended bolus amount. The patient can then decide whether or not to activate the bolus, whether or not to modify the nutritional characteristics of the meal, whether or not to modify the bolus amount, etc. Thus, the system can leverage predictive algorithms to exploit a patient's recurring behaviors or habits to lower the patient burden while maintaining safe glucose levels.

In one or more embodiments, the subset of the patient's historical event data associated with historical instances of the event(s) classified into or mapped to the predicted event ontology may be analyzed to calculate, determine, or otherwise identify representative values for one or more attributes for the event(s) associated with that event ontology. For example, a probable amount of carbohydrates associated with a meal predicted to occur based on the detected real-time sensor data ontology may be calculated by averaging the historical amounts of carbohydrates associated with the patient's past meals that were mapped or classified to the particular event ontology that is correlated with the current sensor data ontology. As another example, an estimated duration of an exercise event predicted to occur based on the real-time sensor data ontological combination may be calculated by averaging the historical exercise durations associated with the patient's past exercise events that were mapped or classified to the particular event ontology that is correlated with the current sensor data ontology. In this regard, historical event data associated with the predicted event ontology may be averaged or otherwise combined to obtain representative values for various event attributes consistent with the patient's historical behavior given the current sensor data ontology.

In one or more exemplary embodiments, in response to detecting a current, real-time sensor data state corresponding to a sensor data ontology that is predictive of occurrence of a particular event ontology, indication of the predicted event(s) and the representative value(s) corresponding to the predicted attributes of the predicted event(s) are transmitted or otherwise provided to the patient's infusion device (e.g., medical device 502) to initiate an action or otherwise alter operation of the infusion device to account for the predicted occurrence of the event(s). For example, in response to detecting a sensor data ontology that is predictive of occurrence of a meal, the SDK or another client application 508 at the patient's client device 508 may transmit or otherwise provide a meal indication to the patient's infusion device 502 along with an estimated amount of carbohydrates and/or other nutritional attributes associated with the meal to the infusion device 502, which, in turn adjusts autonomous operation of the infusion device 502 to account for the predicted meal without the patient manually announcing a meal or other patient burdens. For example, the infusion device 502 may prospectively adjust closed-loop operation to account for a predicted meal with the predicted meal attributes as described in U.S. Patent Pub. No. 2018/0174675. While the subject matter is described in the context of the SDK or client application analyzing the sensor data state in real-time for potential mapping to an event ontology, in other embodiments, the SDK or client application may continually upload the current or real-time sensor data to a remote device in real-time, with the remote device detecting the correlated event ontology, determining representative attributes for the predicted event(s), and transmitting or otherwise providing indicia of the predicted event(s) and predicted event attribute(s) to the patient's client device or medical device, as appropriate.

One exemplary method of monitoring a physiological condition of a patient involves obtaining, at an electronic device associated with the patient, real-time data associated with the patient from a plurality of different data sources coupled to the electronic device (e.g., passively collected sensor data), predicting occurrence of an event in response to detecting when the real-time data corresponds to an ontological state correlative to an event ontology (e.g., by mapping the current sensor data to a sensor data ontology correlative to an event ontology), identifying a representative value for an attribute associated with the event ontology based on a subset of historical event data associated with the patient corresponding to the event ontology (e.g., a probable carbohydrate amount determined by averaging carbohydrate amounts associated with historical meal events mapped to the event ontology), and in response to predicting the occurrence of the event, initiating an action in response to the predicted event, wherein the action is influenced by the representative value for the attribute associated with the event ontology (e.g., prospectively adjusting closed-loop control of the patient's glucose level using the probable carbohydrate amount).

By virtue of the subject matter described herein, burdens on patient's may be reduced by leveraging the predictive and recurrent nature of an individual patient's behavior. While the subject matter may be described herein primarily in the context of meal prediction (or a prediction of a meal time), the subject matter described may be implemented in an equivalent manner to predict the content of a meal, predict the timing or occurrence of exercise, predict the duration and/or intensity of exercise, predict the timing or occurrence of sleep, predict the duration of sleep, and the like.

For the sake of brevity, conventional techniques related to glucose sensing and/or monitoring, machine learning and/or artificial intelligence, pharmodynamic modeling, and other functional aspects of the subject matter may not be described in detail herein. In addition, certain terminology may also be used in the herein for the purpose of reference only, and thus is not intended to be limiting. For example, terms such as “first,” “second,” and other such numerical terms referring to structures do not imply a sequence or order unless clearly indicated by the context. The foregoing description may also refer to elements or nodes or features being “connected” or “coupled” together. As used herein, unless expressly stated otherwise, “coupled” means that one element/node/feature is directly or indirectly joined to (or directly or indirectly communicates with) another element/node/feature, and not necessarily mechanically.

While at least one exemplary embodiment has been presented in the foregoing detailed description, it should be appreciated that a vast number of variations exist. It should also be appreciated that the exemplary embodiment or embodiments described herein are not intended to limit the scope, applicability, or configuration of the claimed subject matter in any way. For example, the subject matter described herein is not limited to the infusion devices and related systems described herein. Moreover, the foregoing detailed description will provide those skilled in the art with a convenient road map for implementing the described embodiment or embodiments. It should be understood that various changes can be made in the function and arrangement of elements without departing from the scope defined by the claims, which includes known equivalents and foreseeable equivalents at the time of filing this patent application. Accordingly, details of the exemplary embodiments or other limitations described above should not be read into the claims absent a clear intention to the contrary.

What is claimed is:

1. A method of monitoring a physiological condition of a patient, the method comprising:
obtaining a predictive association model associated with the patient, wherein the predictive association model is determined based on historical data associated with the patient and comprises a plurality of associations;
obtaining real-time data associated with the patient from a plurality of different data sources;
determining a current state of the patient based at least in part on the real-time data;
predicting occurrence of an event when the current state of the patient corresponds to an association of the plurality of associations; and
in response to predicting the occurrence of the event, initiating an action in response to the predicted event.

2. The method of claim 1, wherein:
each association comprises a subset of a plurality of patient data variable states;
determining the current state comprises determining a current state value for each of the plurality of patient data variable states using the real-time data, resulting in a plurality of current state values; and
detecting when the current state of the patient corresponds to the respective association comprises detecting when a subset of the plurality of current state values matches state values associated with the subset of the plurality of patient data variable states associated with the respective association.

3. The method of claim 1, wherein:
each association comprises a subset of categorical state values for a subset of a plurality of patient data variable states; and
detecting when the current state of the patient corresponds to the respective association comprises:
transforming the real-time data into respective categorical state values for the plurality of patient data variable states; and
detecting when the respective categorical state values for the plurality of patient data variable states includes the subset of categorical state values for the subset of the plurality of patient data variable states associated with the respective association.

4. The method of claim 1, wherein:
obtaining the real-time data comprises obtaining location data associated with the patient from a sensing arrangement;
determining a current location state of the patient based at least in part on the location data; and
the current state of the patient corresponds to the respective association when the current location state of the patient matches a location state associated with the respective association.

5. The method of claim 3, further comprising determining a current amount of elapsed time associated with the current location state based at least in part on the location data, wherein:
detecting when the current state of the patient corresponds to the respective association comprises detecting when the current location state of the patient matches the location state associated with the respective association and the current amount of elapsed time associated with the current location state matches an amount of elapsed time state associated with the respective association.

6. The method of claim 5, further comprising:
transforming the location data into a first categorical location state of a plurality of categorical location states; and
transforming the current amount of elapsed time into a second categorical location duration state of a plurality of categorical location duration states, wherein detecting when the current state of the patient corresponds to the respective association comprises detecting when a combination of the first categorical location state and the second categorical location state matches the respective association.

7. The method of claim 1, further comprising:
generating a table corresponding to the historical data associated with the patient;
transforming entries in the table from real values to categorical values for a plurality of fields of patient data;
transforming the entries in the table from the categorical values for the plurality of fields of patient data into Boolean values for respective categories for respective fields of the plurality of fields of patient data; and
determining the predictive association model based on relationships between respective categorical values for respective subsets of the plurality of fields of patient data and historical occurrences of the event.

8. The method of claim 1, wherein:
each association comprises a respective subset of Boolean values for a respective subset of plurality of fields of patient data; and
predicting the occurrence of the event comprises:
transforming the real-time data into corresponding Boolean values for respective fields of the plurality of fields of patient data; and
detecting when the corresponding Boolean values for respective fields includes the respective subset of Boolean values for the respective subset of plurality of fields of patient data associated with the respective association.

9. The method of claim 1, wherein:
predicting the occurrence of the event comprises predicting consumption of a meal; and
initiating the action comprises automatically generating a user notification indicative of the predicted meal.

10. The method of claim 1, wherein:
predicting the occurrence of the event comprises predicting consumption of a meal; and
initiating the action comprises automatically recommending a bolus dosage of fluid.

11. The method of claim 1, wherein:
predicting the occurrence of the event comprises predicting consumption of a meal; and
initiating the action comprises automatically adjusting operation of an infusion device to deliver insulin in a manner that is influenced by the predicted meal.

12. A method of monitoring a physiological condition of the patient in connection with operation of an infusion device capable of delivering fluid to the patient, the fluid influencing the physiological condition of the patient, the method comprising:
predicting an occurrence of an event based at least in part on real-time data associated with the patient using a predictive association model associated with the patient when a current state of the patient indicated by the real-time data matches a patient state associated with a respective contextual state association of a plurality of contextual state associations associated with the predictive association model; and
in response to the predicted occurrence of the event, automatically initiating one or more actions influenced by the event.

13. The method of claim 12, wherein:
predicting the occurrence of the event comprises predicting consumption of a meal by the patient; and
automatically initiating the one or more actions comprises automatically adjusting operation of an actuation arrangement of the infusion device to deliver the fluid to the patient in a manner that is influenced by the meal.

14. The method of claim 12, wherein:
predicting the occurrence of the event comprises predicting consumption of a meal by the patient; and
automatically initiating the one or more actions comprises automatically generating a user notification including a recommended bolus dosage of the fluid to be delivered by the infusion device in response to the meal.

15. The method of claim 12, wherein:
each contextual state association comprises a subset of categorical state values for a subset of a plurality of patient data variable states; and
predicting the occurrence comprises:
transforming the real-time data into current categorical state values for the plurality of patient data variable states; and
detecting when the current categorical state values for the plurality of patient data variable states includes the subset of categorical state values for the subset of the plurality of patient data variable states associated with the respective contextual state association.

16. The method of claim 12, wherein:
each contextual state association comprises a respective subset of Boolean values for a respective subset of plurality of fields of patient data; and
predicting the occurrence of the event comprises:
transforming the real-time data into corresponding Boolean values for respective fields of the plurality of fields of patient data; and
detecting when the corresponding Boolean values for respective fields includes the respective subset of Boolean values for the respective subset of plurality of fields of patient data associated with the respective contextual state association.

17. A system comprising:
a database to maintain historical data associated with a patient;
a server in communication with the database to transform real values of the historical data to categorical values for a plurality of fields of patient data, and determine a predictive association model for the patient based on relationships between respective categorical values for respective subsets of the plurality of fields of patient data and historical occurrences of an event, wherein the predictive association model comprises a plurality of contextual state associations and each contextual state association comprises a respective subset of the categorical state values for a respective subset of the plurality of fields of patient data; and
a device associated with the patient to obtain the predictive association model from the server via a network, predict occurrence of the event when real-time data associated with the patient corresponds to a respective contextual state association of the plurality of contextual state associations associated with the predictive association model, and initiate an action in response to predicting the event.

18. The system of claim 17, wherein the event comprises a meal and the device initiates the action by generating a user notification indicative of the meal.

19. The system of claim 17, wherein the event comprises a meal and the device initiates the action by operating an actuation arrangement of an infusion device to deliver a bolus dosage of fluid to the patient.

20. The system of claim 18, further comprising a sensing arrangement communicatively coupled to the device to provide measurement data associated with the patient to the device, wherein the device predicts the occurrence of the event when the measurement data associated with the patient corresponds to the respective contextual state association.
